(19) United States
(12) Patent Application Publication
(10) Pub. No.: US 2002/0133131 A1
(43) Pub. Date: Sep. 19, 2002

(54) Absorbent material incorporating synthetic fibers and process for making the material

(76) Inventors: Krishnakumar Rangachari, Savannah, GA (US); Kays Chinai, Simons Island, GA (US)

Correspondence Address: ROCKEY, MILNAMOW & KATZ, LTD., Two Prudential Plaza - Suite 4700, 180 North Stetson Avenue, Chicago, IL, 60601, US

(21) Appl. No.: 09/757,214

(22) Filed: Jan. 9, 2001

(51) Int. Cl. A61F 13/15

(52) U.S. Cl. 604/370; 604/372; 604/374; 604/368; 604/379

(57) ABSTRACT

A soft, high density, absorbent material is provided with improved characteristics. A process is provided for making the absorbent material. A web is formed from material that includes a mixture of cellulosic fibers and synthetic polymer fibers. Then, the web is preferably compacted and embossed at an elevated temperature to further increase the web density and preferably to also create liquid-stable bonds between the synthetic polymer fibers and the cellulosic fibers in spaced-apart regions of the web.

### STATEMENT REGARDING FEDERALLY-SPONSORED RESEARCH OR DEVELOPMENT

Not applicable.

### REFERENCE TO MICROFICHE APPENDIX

Not applicable.

### TECHNICAL FIELD

This invention relates to absorbent materials and to a process for making absorbent materials to be used as absorbent cores in articles such as disposable diapers, feminine hygiene products and incontinence devices. More particularly, the present invention relates to improved absorbent materials that are high density, strong, soft materials with superior absorption properties, especially fluid acquisition capability.

### BACKGROUND OF THE INVENTION AND TECHNICAL PROBLEMS POSED BY THE ART

Disposable absorbent articles, such as diapers, feminine hygiene products, adult incontinence devices and the like have found widespread acceptance. To function efficiently, such absorbent articles must quickly absorb body fluids, distribute those fluids within and throughout the absorbent article and be capable of retaining those body fluids with sufficient energy to dry the body surface when placed under loads. In addition, the absorbent article should be sufficiently soft and flexible so as to comfortably conform to body surfaces and provide close fit for lower leakage.

While the design of individual absorbent articles varies depending upon use, there are certain elements or components common to such articles. The absorbent article contains a liquid pervious top sheet or facing layer, which facing layer is designed to be in contact with a body surface. The facing layer is made of a material that allows for the substantially unimpeded transfer of fluid from the body into the core of the article. The facing layer should not absorb fluid per se and, thus, should remain dry. The article further contains a liquid impervious back sheet or backing layer disposed on the outer surface of the article and which layer is designed to prevent the leakage of fluid out of the article.

Disposed between the facing layer and backing layer is an absorbent member referred to in the art as an absorbent core or panel. The function of the absorbent core is to absorb and retain body fluids entering the absorbent article through the facing layer. Because the origin of body fluids is often localized, it is desirable to provide means for distributing fluid throughout the dimensions of the absorbent core to make full use of all the available absorbent material. This is typically accomplished either by providing a distribution member disposed between the facing layer and absorbent core and/or altering the composition of the absorbent core per se.

Fluid can be distributed to different portions of the absorbent core by means of an optional transition layer, transfer layer, or acquisition layer disposed between the facing layer and core. The purpose of the acquisition layer is to facilitate lateral spreading of the fluid, and further to rapidly transfer and distribute the fluid to the absorbent core. Although a separate acquisition layer can function generally satisfactory in performing the above-described functions, the incorporation of a separate acquisition layer in an absorbent material product complicates the structure and requires additional manufacturing steps. This also necessarily increases the cost of the absorbent material product. Accordingly, it would be desirable in some applications to provide an absorbent material product which does not employ such a separate acquisition layer and yet which has improved acquisition capability. Further, it would be desirable to provide such an improved absorbent material product with increased acquisition capability without significantly increasing the stiffness of the product. It would be desirable to provide such an improved absorbent material product with a composition that results in a soft and supple product.

A conventional absorbent core is typically formulated of a cellulosic wood pulp fiber matrix, which is capable of absorbing large quantities of fluid. Absorbent cores can be designed in a variety of ways to enhance fluid absorption and retention properties. By way of example, the fluid retention characteristics of absorbent cores can be greatly enhanced by disposing superabsorbent materials in amongst fibers of the wood pulp. Superabsorbent materials are well known in the art as substantially water-insoluble, absorbent polymeric compositions that are capable of absorbing large amounts of fluid in relation to their weight and forming hydrogels upon such absorption. Absorbent articles containing blends or mixtures of pulp and superabsorbents are known in the art.

The distribution of superabsorbents within an absorbent core can be uniform or non-uniform. By way of example, that portion of an absorbent core proximate to the backing layer (farthest away from the wearer) can be formulated to contain higher levels of superabsorbent than those portions of the core proximate the facing or acquisition layer. By way of further example, that portion of the core closest to the site of fluid entry (e.g., acquisition zone) can be formulated to transport (wick) fluid into surrounding portions of the core (e.g., storage zone).

In addition to blending pulp with superabsorbent material, a variety of other means for improving the characteristics of pulp have been described. For example, pulp boards can be more easily defiberized by using chemical debonding agents (see, e.g., U.S. Pat. No. 3,930,933). In addition, cellulose fibers of wood pulp can be flash-dried prior to incorporation into a composite web absorbent material (see, e.g., U.K. Patent Application GB 2272916A published on Jun. 1, 1994). Still further, the individualized cellulosic fibers of wood pulp can be cross-linked (see, e.g., U.S. Pat. Nos. 4,822,453; 4,888,093; 5,190,563; and 5,252,275). All of these expedients have the disadvantage of requiring the wood pulp manufacturer to perform time-intensive, expensive procedures during the wood pulp preparation steps. Thus, use of these steps results in substantial increases in the cost of wood pulp.

Although all of the above-described treatment steps have been reported to improve the absorption characteristics of pulp for use as absorbent cores, there are certain disadvantages associated with such treatments. By way of example, the manufacturer of the end use absorbent article (e.g. feminine hygiene product or diaper) typically procures wood pulp in the form of a sheet from a wood pulp manufacturer. The end use article manufacturer must then fluff the fibers in the wood pulp sheet so as to detach the individual fibers bound in that pulp sheet. Typically, pulp has a low moisture content, and this causes the individual fibers to be relatively brittle—resulting in fine dust due to fiber breakage during fluffing operations. If the pulp manufacturer performs such fluffing prior to shipment to the absorbent article maker, the transportation costs of the pulp are increased. At least one pulp manufacturer has attempted to solve this problem by producing flash-dried pulp without chemical bonding agents in a narrow range of basis weights and pulp density (see U.S. Pat. No. 5,262,005). However, even with this process, the manufacturer of the absorbent article must still process the pulp after purchase.

There have been numerous attempts by the manufacturers of absorbent materials to produce highly absorbent, strong, soft core materials. U.S. Pat. No. 4,610,678 discloses an air-laid material containing hydrophilic fibers and superabsorbent material, wherein the material is air-laid in a dry state and compacted without the use of any added binding agents. Such material, however, has low integrity and suffers from shake-out or loss of substantial amounts of superabsorbent material. U.S. Pat. No. 5,516,569 discloses that superabsorbent material shake-out can be reduced in air-laid absorbents by adding significant amounts of water to material during the air-laying process. The resultant material, however, is stiff, of low density and has a high water content (greater than about 15 weight percent). U.S. Pat. No. 5,547,541 discloses that high density air-laid materials containing hydrophilic fibers and superabsorbent material can be made by adding densifying agents to the material. The use of such agents, however, increases the production cost of the material.

U.S. Pat. No. 5,562,645 discloses low density absorbent materials (density less than 0.25 g/cc). The use of such low density, bulky materials increases the cost of transportation and handling. U.S. Pat. No. 5,635,239 discloses an absorbent material that contains two complex forming agents that interact when wetted to form a complex. The complex forming agents are polymeric olefins. European Patent Application No. EP 0763364 A2 discloses absorbent material that contains cationic and anionic binders that serve to hold the superabsorbent material within the material. The use of such agents and binders increases the cost of making the absorbent material and poses a potential environmental hazard.

The U.S. Pat. No. 2,955,641 and 5,693,162 disclose (1) the application of steam to absorbent material to increase the moisture content of the absorbent material, and (2) compressing the absorbent material. The U.S. Pat. No. 5,692,162 also discloses the use of hot calendering rolls (which may be patterned) to form a densified structure, and the use of thermoplastic and thermo-setting resins suitable for thermal bonding.

U.S. Pat. No. 5,919,178 discloses a process for producing an absorbent structure having an intermediate layer containing superabsorbent material sandwiched between two absorbing layers wherein the bottom layer can be a tissue. The patent discloses that when tissue is used as the upper or lower layer, the moisture content of the tissue shall be 20%-70% (by, for example, spraying the tissue with moisture immediately prior to calendaring at a line pressure of 100-200 kg/cm and a temperature of 120° C.-250° C. to compress the web to a density of 0.1 g/cm³ to produce a pulp mat thickness of 1 mm-4 mm).

Some absorbent structures have been developed to include fibers which have been formed from one or more thermoplastic polymers. The published international application PCT/US99/29468, publication WO 00/34567, discloses the use of a bicomponent synthetic thermoplastic fiber which includes a first polymer component formed as a core surrounded by a sheath of a second polymer component. Typically, portions of thermoplastic fibers are melted to form a tacky skeletal structure. In conventional products employing a bicomponent fiber having a sheath surrounding a core, the polymer material comprising the sheath melts at a temperature lower than that of the core. The melted portions of the sheath can then, upon cooling, form thermal bonds with other thermoplastic fibers. Bonds can also be formed between the plastic and the pulp fibers according to WO 00/34567.

Prior art absorbent material products that employ thermally bonded thermoplastic fiber webs are typically not very soft because the prior art thermal bonding process imparts a degree of increased rigidity to the structure. Some investigators have reported that no wetting or adhesion of the molten thermoplastic fiber to the cellulosic fiber surface can be observed (K. Kohlhammer, Dr. Klaus. “SELF-CROSS LINKABLE POWDER RESINS IN AIR LAID NONWOVENS,” NONWOVENS WORLD, June-July 2000, MTS Publications, Kalamazoo, Mich., U.S.A.). Further, conventionally thermal bonded webs can have dust problems and Tinting problems.

While such prior art structures employing thermoplastic fibers may provide an increased bonding of the absorbent core, or of an acquisition layer to an absorbent core, it would be desirable to provide an absorbent material with improved absorption characteristics, such as improved fluid acquisition characteristics, while at the same time providing a material which still remains relatively soft and supple and which does not have a significant increase in rigidity.

Many prior art absorbent material structures that have a low density and that are thick have functioned relatively well to absorb fluid, but the low density and thickness of such prior art structures has obvious disadvantages. It would be desirable to provide an improved absorbent core material which would have a higher density and be relatively thin while at the same time remaining soft and supple and providing good absorbency characteristics.

Such an improved absorbent material structure should also preferably accommodate manufacturing and subsequent handling with a reduced tendency to break or fall apart. Such an improved structure should have sufficient tensile strength and integrity to be functional, both in the dry condition and in the wet condition. It would also be advantageous to provide such an improved absorbent material with an integral structure that promotes, and enhances, acquisition of fluid into the structure.

There continues to be a need in the art for an improved process for making an absorbent material which has good fluid acquisition capability and which satisfies the absorbency, strength and softness requirements needed for use as an absorbent core in disposable absorbent articles and which also provides time and cost savings to both the pulp manufacturer and the manufacturer of the absorbent article.

It would be desirable to provide an improved process for efficiently manufacturing such an improved absorbent material at reduced cost and with an improved capability for consistently producing the material with predetermined characteristics of absorbency, strength, softness, etc.

### BRIEF SUMMARY OF THE INVENTION

The present invention provides an absorbent material which may be characterized as having a relatively high density so that an absorbent core made from the material is relatively thin. The material exhibits good absorbency characteristics, including good fluid acquisition characteristics. Further, the absorbent material of the present invention, although having a relatively high density, is relatively soft and supple. Also, the absorbent material of the present invention is relatively strong and has good integrity and tensile strength so as to withstand manufacturing and subsequent handling and use.

The material of this invention has superior absorptive properties. The absorbent material can be used to make absorbent articles, such as a diaper, a feminine hygiene product, or an incontinence device. The absorbent material comprises a compacted web of cellulosic fibers and synthetic polymer fibers, and the web has a Gurley Stiffness of less than about 1500 milligrams, preferably less than about 1200 milligrams. In one preferred form, the web includes superabsorbent material, but the web is substantially free of added chemical binders.

In one preferred form of the invention, at least some of the synthetic polymer fibers and cellulosic fibers are joined by liquid stable bonds.

In one preferred form of the invention, at least the major portion of the total surface area defined on the exteriors of the synthetic polymer fibers has not been melted and resolidified.

In one preferred form of the invention, the web has a density between about 0.25 grams per cubic centimeter and about 0.50 grams per cubic centimeter, and the web has a basis weight between about 100 grams per square meter and about 650 grams per square meter.

The process for making the absorbent material of the invention includes first forming a web of cellulosic fibers and synthetic polymer fibers.

The web is moved between a pair of heated rolls to compact the web while maintaining each of the rolls at temperatures to form liquid stable bonds that (1) are located at least between the cellulosic fibers and the synthetic polymer fibers, and (2) are insufficient to create a web stiffness of more than about 1500 milligrams.

In a preferred form of the process, the web is produced without the use of heater ovens, and also includes superabsorbent material, but is substantially free of added chemical binders.

In a preferred form of the process, the web is moved at a selected speed between a pair of heated rolls which are embossed or have a surface pattern and which compact the web under a selected compaction load to a density of between about 0.25 grams per cubic centimeter and 0.50 grams per cubic centimeter while maintaining each of the rolls at temperatures which are insufficient at the selected speed of web movement and the selected compaction load to melt a major portion of the total surface area defined by the exteriors of the synthetic polymer fibers whereby the web Gurley Stiffness of the compacted web is less than about 1500 milligrams, preferably less than about 1200 milligrams.

Numerous other advantages and features of the present invention will become readily apparent from the following detailed description of the invention, from the claims, and from the accompanying drawings.

### BRIEF DESCRIPTION OF THE DRAWINGS

In the drawings, which form a portion of the specification:

FIG. 1 is a greatly enlarged, fragmentary cross-sectional view of a web or sheet of a first embodiment of absorbent material of the present invention, and in FIG. 1 the height or thickness of portions of the illustrated structure have been exaggerated for ease of illustration, and it should be understood that FIG. 1 is not necessarily drawn to scale with respect to the thickness of the various portions;

FIG. 2 is a greatly enlarged, fragmentary cross-sectional view of a web or sheet of a second embodiment of absorbent material of the present invention, and in FIG. 2 the height or thickness of portions of the illustrated structure have been exaggerated for ease of illustration, and it should be understood that FIG. 2 is not necessarily drawn to scale with respect to the thickness of the various portions;

FIG. 3 is a simplified, diagrammatic view of an apparatus illustrating a preferred process for making the improved material of the present invention;

FIG. 4 is a simplified, schematic illustration of a device for measuring the wicking properties of absorbent material;

FIG. 5 is a representative plot or graph of fluid absorption versus distance obtained in a 45 degree wicking test that can be performed on the device illustrated in FIG. 4;

FIG. 6 is a simplified, schematic illustration of apparatus for producing an incomplete form, or first stage form, of the absorbent material of the present invention;

FIG. 7 is a view of the apparatus in FIG. 6 showing a second stage in the production of the absorbent material;

FIG. 8 is a simplified, schematic illustration of apparatus for completing the production of the absorbent material, the first stage and second stage production of which is illustrated in FIGS. 6 and 7;

FIG. 9 is a simplified, schematic illustration of another apparatus for effecting the first stage of the production of absorbent material of the present invention;

FIG. 10 is a simplified, schematic illustration of apparatus for effecting the final stage of the production of the material after having completed the first stage illustrated in FIG. 9;

FIG. 11 illustrates a first embossing pattern for the surface of the absorbent material of the present invention;

FIG. 12 illustrates a second embossing pattern for the surface of the absorbent material of the present invention;

FIG. 13 illustrates a third embossing pattern for the surface of the absorbent material of the present invention;

FIG. 14 is an enlarged, diagrammatic illustration of a portion of absorbent material made according to the present invention by a process employing the embossing pattern 2 illustrated in FIG. 12, and the portion of the material illustrated in FIG. 14 corresponds to the location of the portion of material shown relative to the embossing pattern located within the circle designated generally by the reference number 300 in FIG. 12;

FIG. 15 is a scanning electron microscope photomicrograph of a portion of absorbent material according to the present invention;

FIG. 16 is a view similar to FIG. 15, but FIG. 16 shows a different portion of the material; and

FIG. 17 is a scanning electron microscope photomicrograph of absorbent material employed in a conventional product.

### DETAILED DESCRIPTION

The present invention provides an improved absorbent material that is particularly well-suited for use as cores in absorbent articles such as diapers, feminine hygiene products, incontinence devices, and the like. The absorbent material can also be used as an absorbent core in any device used for absorbing body exudates (e.g., urine, breast milk, blood, serum). Thus, the absorbent material can be incorporated into breast pads for nursing mothers or used as absorbent material in surgical drapes (e.g., towels) or wound dressings.

The preferred form of the absorbent material includes a blend of cellulosic fibers, synthetic polymer fibers, and superabsorbent material. Preferably, these materials are air laid onto a carrier layer (e.g., tissue web). The absorbent material has a unique combination of suppleness, strength, and absorbency characteristics that makes it particularly suitable for use in absorbent articles. The absorbent material can be used directly by a manufacturer of the absorbent article without the need for any additional processing by that manufacturer other than cutting or folding the absorbent material to the desired size and shape for the absorbent article.

Another aspect of the present invention is an improved process which can be used to make an absorbent material that is soft, that is thin, and that has relatively high density. The preferred form of the process is effected without the use of expensive heater ovens and does not require the use of chemical binders, adhesives, or the like. The absorbent material has enough integrity (strength) to be further processed on conventional disposable product manufacturing equipment without significant fiber breakage.

With reference to the composition of an existing material containing an added substance, the phrase “weight percent” of the substance as used herein means the weight of the added substance divided by the total, combined weight of the added substance and original material (as determined under ambient conditions) and multiplied by 100. By way of example, an absorbent material product containing 10 weight percent of added superabsorbent material means that there are 10 grams of superabsorbent material in a 100 gram specimen containing both the initial absorbent material and the added superabsorbent material.

Cellulosic fibers that can be used in the process of the present invention are well known in the art and include wood pulp, cotton, flax, and peat moss. Wood pulp is preferred. Pulps can be obtained from mechanical or chemi-mechanical, sulfite, kraft, pulping reject materials, organic solvent pulps, etc. Both softwood and hardwood species are useful. Softwood pulps are preferred. It is not necessary to treat cellulosic fibers with chemical debonding agents, cross-linking agents and the like for use in the absorbent material.

As discussed above, a preferred cellulosic fiber for use in the present material is wood pulp. Wood pulp prepared using a process that reduces the lignin content of the wood is preferred. Preferably, the lignin content of the pulp is less than about 16 percent. More preferably, the lignin content is less than about 10 percent. Even more preferably, the lignin content is less than about 5 percent. Most preferably, the lignin content is less than about 1 percent. As is well known in the art, lignin content is calculated from the Kappa value of the pulp. The Kappa value is determined using a standard, well known test procedure (TAPPI Test 265-cm 85). The Kappa value of a variety of pulps was measured and the lignin content calculated using the TAPPI Test 265-cm 85.

For use in the process of the present invention, cellulosic fibers are preferably obtained from wood pulp having a Kappa value of less than about 100. Even more preferably, the Kappa value is less than about 75, 50, 25 or 10. Most preferably, the Kappa value is less than about 2.5.

There are certain other characteristics of wood pulp that make it particularly suitable for use in an absorbent material. Cellulose in most wood pulps has a high relative crystallinity (greater than about 65 percent). In the preferred form of the material of the present invention, the use of wood pulp with a relative crystallinity of less than about 65 percent is preferred. More preferably, the relative crystallinity is less than about 50 percent. Most preferably, the relative crystallinity is less than about 40 percent. Also, pulps having an increased fiber curl value are preferred.

Means for treating pulps so as to optimize these characteristics are well known in the art. By way of example, treating wood pulp with liquid ammonia is known to decrease relative crystallinity and to increase the fiber curl value. Flash drying is known to increase the fiber curl value of pulp and to decrease crystallinity. Cold caustic treatment of pulp also increases fiber curl and decreases relative crystallinity. Chemical cross-linking is known to decrease relative crystallinity. For one form of the material of the present invention, it is preferred that the cellulosic fibers used to make the absorbent material by the process of this invention are obtained at least in part using cold caustic treatment or flash drying.

A description of the cold caustic extraction process can be found in commonly owned U.S. patent application Ser. No. 08/370,571, filed on Jan. 18, 1995, which application is a continuation-in-part application of U.S. patent application Ser. No. 08/184,377, filed on Jan. 21, 1994, now abandoned. The disclosures of these two U.S. patent applications are incorporated in their entirety herein by reference thereto.

Briefly, a caustic treatment is typically carried out at a temperature less than about 60° C., but preferably at a temperature less than 50° C., and more preferably at a temperature between about 10° C. and about 40° C. A preferred alkali metal salt solution is a sodium hydroxide solution newly made up or as a solution by-product in a pulp or paper mill operation, e.g., hemicaustic white liquor, oxidized white liquor and the like. Other alkali metals such as ammonium hydroxide and potassium hydroxide and the like can be employed. However, from a cost standpoint, the preferable salt is sodium hydroxide. The concentration of alkali metal salts is typically in a range from about 2 to about 25 weight percent of the solution, and preferably from about 6 to about 18 weight percent. Pulps for high rate, fast absorbing applications are preferably treated with alkali metal salt concentrations from about 10 to about 18 weight percent.

As is well known in the art, flash drying is a method for drying pulp in which pulp is partially dewatered, fiberized, and fed into a stream of hot air which causes the moisture contained in the pulp to be flashed off. Briefly, the pulp, initially at a consistency of 30-50% (containing 50-70% water), is conveyed directly into a fluffer (e.g., a disk refiner) where mechanical action is used to fiberize (break up and separate) and disperse the fibers for the flash drying system. Once discharged from the fluffer device, the fiberized pulp is fed into a flash drying system. The drying system itself is made up of two stages, each of which consists of two drying towers. The fiber is conveyed through the drying towers by high velocities of hot air. The inlet air temperature for the first stage is approximately 240-260° C. while the inlet air temperature for the second stage is approximately 100-120° C. Following each drying stage, the pulp and hot air are then conveyed into a cyclone separator, where the hot air, now containing moisture evaporated from the pulp, is exhausted vertically.

In a typical, small scale system, exhaust temperatures for the first stage of such a drying system are approximately 100-120° C., and the exhaust temperatures for the second stage are approximately 90-100° C. At the same time, a material-handling fan draws the pulp fibers through the cyclone cone and on to the next part of the system. Finally, following the second stage cyclone separator, the dried pulp is passed through a cooling stage consisting of a cooling fan which conveys ambient air, and is then passed through a final cooling cyclone separator. The residence time for the entire system, including both drying stages, cyclone separation, and cooling, is approximately 30-60 seconds at the feed rate used (1.5 kg of dry material per minute which is a feed rate typical of a small scale machine). Larger scale. conventional flash drying systems typically have higher feed rates.

A downside to producing flash dried fiber using the type of system described above is the production of localized fiber bundles in the final product. Fiber bundles are formed during the fiberization of the pulp by mechanical action within the fluffer device. The system above uses a disk refiner consisting of two grooved, circular plates at a set gap width, in this case 4 mm. One plate is in a fixed position while the other plate is rotated at high speeds. The pulp is fed into the gap between the two plates and the rotation of the plate results in the separation of fibers along the grooves. Unfortunately, as the pulp is fiberized, some of the individual fibers tend to become entangled with one another, forming small bundles consisting of several individual fibers. As these entangled fibers are flash dried and the moisture is removed, the entanglements tighten and harden to form small localized fiber bundles throughout the flash dried pulp. The presence of large numbers of these localized fiber bundles within the final airlaid products produced using the flash dried pulp can have a deleterious effect on the product physical characteristics and performance. The number of localized fiber bundles can be substantially reduced by using cold caustic extracted pulp.

According to one aspect of the process of the present invention (as described hereinafter), the absorbent material of the present invention is manufactured to contain a superabsorbent material. Superabsorbent materials are well known in the art. As used herein, the term “superabsorbent material” means a substantially water-insoluble polymeric material capable of absorbing large quantities of fluid in relation to its weight. The superabsorbent material can be in the form of particulate matter, flakes, fibers and the like. Exemplary particulate forms include granules, pulverized particles, spheres, aggregates and agglomerates. Exemplary and preferred superabsorbent materials include salts of crosslinked polyacrylic acid such as sodium polyacrylate. Superabsorbent materials are commercially available (e.g., from Stockhausen GmbH, Krefeld, Germany). Preferred forms of the absorbent material of the present invention contain from about 0 to about 60 weight percent superabsorbent material and, more preferably from about 20 to about 60 weight percent superabsorbent material.

Preferred Forms of the Absorbent Material

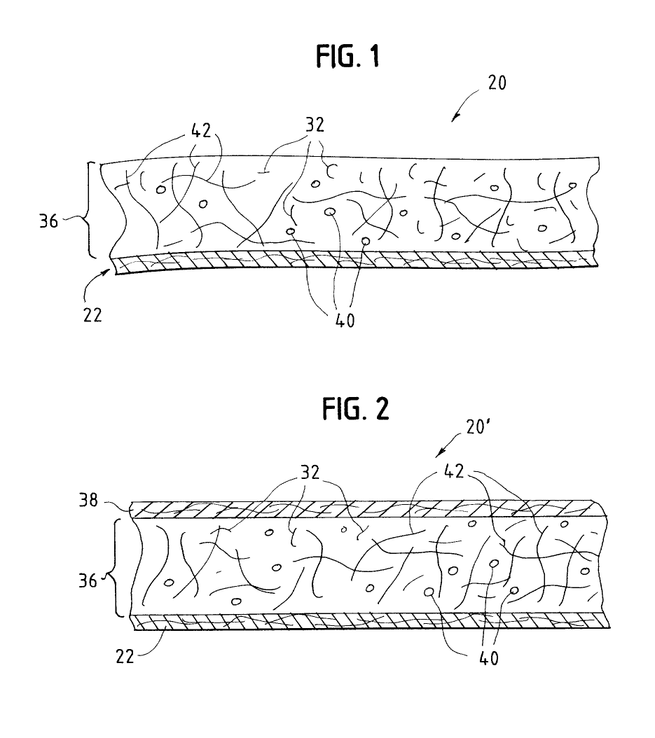

FIG. 1 illustrates one form of an absorbent material of the present invention. The absorbent material is designated in FIG. 1 generally by the reference number 20. The material 20 is typically made by the process of the present invention in a relatively wide sheet that can be provided in sheet form or in a large roll to a manufacturer of absorbent articles.

A typical, preferred thickness of the material is between 0.5 mm and 2.5 mm. Regions of various thickness in the material 20 illustrated in FIG. 1 are not necessarily shown to scale and may in some respects be exaggerated for purposes of clarity and ease of illustration.

The absorbent material 20 illustrated in FIG. 1 includes a primary absorbent portion or core 36 and an optional carrier layer 22. The carrier layer 22 may be, for example, a spunbond, melt blown non-woven consisting of natural or synthetic fibers or may be some other material.

Another, and preferred, material that could be used for the carrier layer 22 is tissue. Suitable tissue materials for use as a carrier layer in absorbent products are well known to those of ordinary skill in the art. Preferably such tissue is made of bleached wood pulp and has an air permeability of about 273-300 CFM (cubic feet minute). The tensile strength of the tissue is such that it retains integrity during formation and other processing of the absorbent material. Suitable MD (machine direction) and CD (cross direction) tensile strengths, expressed in newtons/meter, are about 100-130 and 40-60, respectively. The tissue may be a crepe tissue having a sufficient number of crepes per inch to allow a machine direction elongation of between 20 and 35 percent (as determined by the SCAN P44:81 test method). Tissue for use in air-laying absorbent materials are commercially available (e.g., from Cellu Tissue Corporation, 2 Forbes Street, East Hartford, Conn. 06108, U.S.A., and from Duni AB, Sweden).

A modification of the absorbent material structure 20 illustrated in FIG. 1 is shown in FIG. 2 where the modification is designated generally by the reference number 20′. Regions of various thicknesses of the material 20′ illustrated in FIG. 2 are not necessarily to scale and may in some respects be exaggerated for purposes of clarity and ease of illustration.

The material 20′ illustrated in FIG. 2 includes a primary absorbent portion 36 and a carrier layer 22 which may be identical with the primary absorbent portion 36 and carrier layer 22 in the first embodiment material 20 described above with reference to FIG. 1. The modified embodiment of the absorbent material 20′ in FIG. 2 further includes a top carrier layer or cover layer 38. The cover layer 38 may be tissue or may be another type of material, including, but not limited to, a spunbond melt blown non-woven consisting of natural or synthetic fibers.

Preferably, when a carrier layer, such as tissue layer 22, is used, the tissue layer 22 is lightly embedded into the bottom of the primary absorbent portion 36, and this can be effected during processing with a roll or rolls as described in more detail hereinafter.

The primary absorbent portion 36 of each embodiment of the material 20 and 20′ (FIGS. 1 and 2) includes pulp fibers 32 which, in one preferred form, have a typical average length of about 2.40 mm. In one preferred form of the pulp fibers 32, at least some of the pulp fibers 32 are produced by the above-discussed cold caustic extraction process. This includes treating a liquid suspension of pulp containing cellulosic fibers at a temperature of from about 15° C. to about 60° C. with an aqueous alkali metal salt solution having an alkali metal salt concentration from about 2 weight percent to about 25 weight percent of the solution for a period of time ranging from about 5 minutes to about 60 minutes. The treated pulp cellulosic fibers are then either flash-dried or processed through a hammermill.

The primary absorbent portion 36 (FIGS. 1 and 2) also preferably includes a superabsorbent material of the type previously described and which preferably is provided in the form of superabsorbent granules or particles 40. If desired, the pulp and superabsorbent can be laid down as a homogenous blend or as a heterogeneous blend wherein the level of superabsorbent varies with proximity to the bottom (i.e., the bottom carrier layer 22).

Typically, an absorbent article manufacturer would add a facing layer (i.e., a top sheet or cover stock (not illustrated)) against one side of the absorbent material 20 or 20′, and such a facing layer contacts the skin of the person wearing the article. The upper portion of the material 20 or 20′ next to the facing layer can receive liquid (e.g., menses or urine) in the first moments of discharge through the facing layer. The upper portion of the material 20 should preferably pick up the liquid from the absorbent article facing layer very quickly and distribute the liquid throughout the absorbent portion 36. It would be desirable to provide means for facilitating the lateral spreading of the liquid, especially during second and subsequent discharges of liquid into the absorbent article.

One aspect of the present invention provides an absorbent material with an improved capability for acquiring the liquid and laterally distributing the liquid in the primary absorbent portion or core 36. To this end, the primary absorbent portion or core 36 includes synthetic polymer fibers 42 (FIGS. 1 and 2). The synthetic polymer fibers are preferably longer than the pulp fibers 32. Preferably, the synthetic polymer fibers are between about two and about four times as long as the pulp fibers 32. Whereas pulp fibers may be about 2 millimeters in length, the synthetic polymer fibers may be between about 4 and about 6 millimeters in length, although some synthetic polymer fibers may be shorter and some may be longer.

The synthetic polymer fibers 42 typically have a circular cross section in contrast with the pulp fibers 32 which may have a somewhat rectangular cross section. It is believed that with the present invention it may be preferable, at least in some applications, that the synthetic polymer fibers 42 not be too long (that is not too much longer than 4-6 millimeters) so as to enhance the primary absorbent portion void and loft characteristics as well as wicking capability compared to the use of longer fibers. Synthetic polymer fibers which are about 6 millimeters or less in length may be more likely to be oriented with the lengths of the fibers extending at oblique angles to the general plane defined by the length and width of the primary absorbent portion compared to longer synthetic polymer fibers that would have more of a tendency to lie parallel to the length and width plane of the primary absorbent portion.

In presently preferred forms of the invention material, the synthetic polymer fibers in the absorbent core portion are preferably made from polypropylene or polyethylene terephthalate. The synthetic polymer fibers may all be made from a single synthetic polymer such as polypropylene or polyethylene terephthalate. For example, polypropylene fibers may be provided in a nominal 6 millimeter length at 6.7 dtex in a high crimped condition. (The 6.7 dtex number refers to the weight in grams of 10,000 meters of the fiber.) Absorbent materials made according to the present invention to date have also included polyethylene terephthalate fibers having a nominal length of 6.35 millimeters and 6 dtex in a high crimped condition as well as polyethylene terephthalate fibers having nominal length of 12.7 millimeters and 17 dtex in a high crimped condition.

The present invention also contemplates the use of bicomponent synthetic polymer fibers in the absorbent core portion. One example of bicomponent fibers that is suitable for use in the present invention includes a polypropylene core and a polyethylene sheath and has a nominal length of 6 millimeters and 1.7 dtex.

Preferably, the basis weight of the primary absorbent portion 36 (FIGS. 1 and 2) is between about 100 and about 650 g/m². The basis weight of the carrier layer 22 is typically between about 15 and about 20 g/m², but could be more or less. The basis weight of the cover layer 32 (FIG. 2) is typically between about 10 and about 50 g/m², but could be more or less.

The average density of the absorbent material 20 or 20′ preferably ranges between 0.25 and 0.50 g/cm³. The moisture content of the absorbent material 20 and 20′ after equilibration with the ambient atmosphere is preferably less than about 10% (by weight of the total material weight), is more preferably less than about 8%, and preferably lies in the range of between about 1% and about 8%.

Preferred Production Process

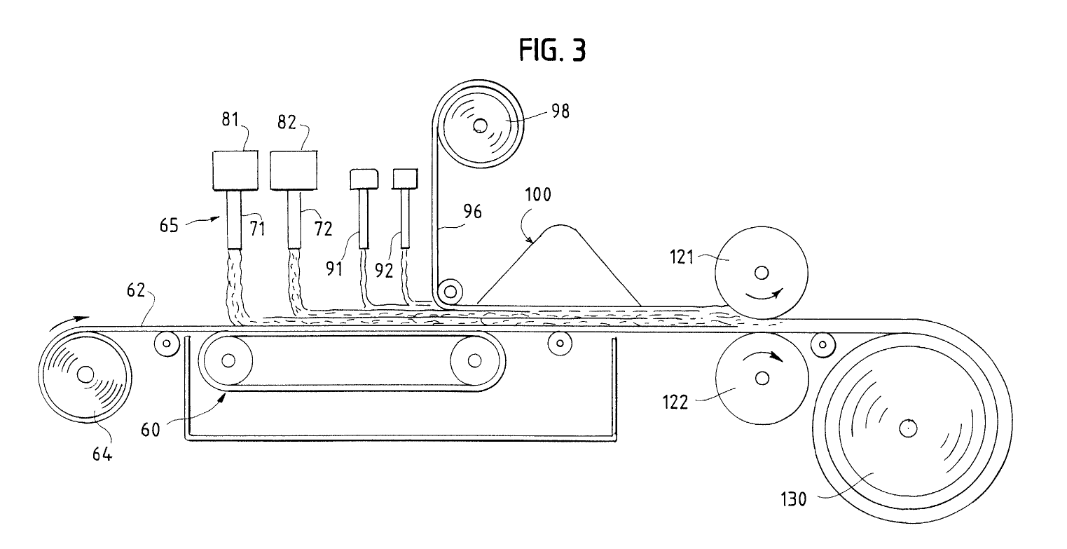

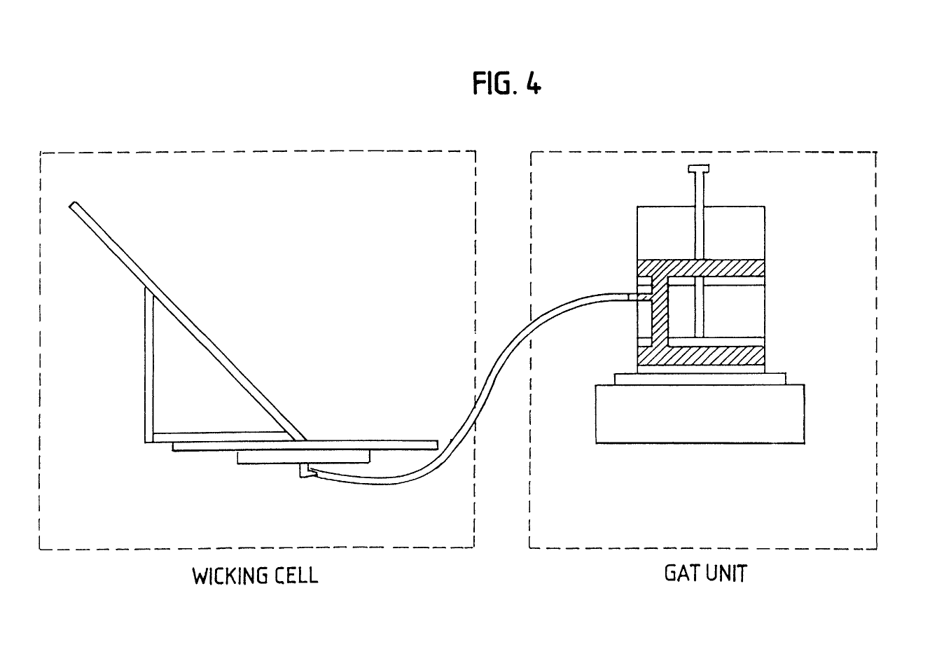

The above-described absorbent materials may be made with the process of the present invention. A presently contemplated, preferred embodiment of the process of the present invention is diagrammatically illustrated in FIG. 3. The illustrated process employs an endless wire, screen, or belt 60 on which the absorbent material components are deposited.

The process permits the optional incorporation of a bottom carrier layer in the absorbent material (e.g., tissue layer 22 in the absorbent material 20 and 20′ described above with reference to FIGS. 1 and 2, respectively). To this end, as shown in FIG. 3, a tissue web 62 is unwound from a tissue web roll 64 and directed over the endless screen 60. A series of forming heads in a forming head station 65 are provided over the endless screen 60. The station 65 includes a first forming head 71 and a second forming head 72. A lesser or greater number of forming heads may be provided.

Cellulosic fibers, which may include 0%-100% of the above-described cold caustic extracted pulp fibers, are processed using a conventional hammermill (not illustrated) to individualize the fibers. The individualized pulp fibers can be blended with synthetic polymer fibers and superabsorbent material (e.g., granules, particles, etc.) in a blending system supplying each forming head. The forming head 71 is connected with a blending system 81, and the forming head 72 is connected with a blending system 82. The pulp fibers, synthetic polymer fibers, and superabsorbent granules or particles can be blended in the blending systems and conveyed pneumatically into the one or more of the forming heads. Alternatively, the pulp fibers, synthetic polymer fibers, and superabsorbent granules or particles can be conveyed separately to one or more forming heads and then blended together in the forming heads. One or more of the forming heads may be operated to discharge only pulp without discharging synthetic polymer fibers or superabsorbent material. Chemical binding agents are not added during fiber processing or during the blending of the cellulosic, pulp fibers with the synthetic polymer fibers and/or superabsorbent material.

The blending and distribution of the materials can be controlled separately for each forming head. For example, in some systems, controlled air circulation and winged agitators in each blending system produce a substantially uniform mixture and distribution (of the pulp and superabsorbent particles and/or synthetic polymer fibers for blending systems 81 and 82).

The superabsorbent particles and synthetic polymer fibers can be either thoroughly and homogeneously blended with the pulp fibers and synthetic fibers throughout the absorbent core portion of the structure being produced, or can be contained only in a specific thickness regions by distributing the superabsorbent particles and/or synthetic polymer fibers to selected forming heads.

If desired, the superabsorbent particles and synthetic polymer fibers can be separately discharged from separate forming heads 91 and 92 (FIG. 3), respectively. In such an optional configuration, the superabsorbent particle forming head 91 and synthetic polymer fiber forming head 92 can be located downstream of the forming heads 71 and 72 as shown or can be located upstream of, or among, the other forming heads (not illustrated). Also, the upstream-downstream order of the forming heads 91 and 92 could be reversed from that shown in FIG. 3. If separate forming heads 91 and 92 are employed for the superabsorbent material and the synthetic polymer fibers, then additional superabsorbent particles and/or synthetic polymer fibers could also still be blended in the blending systems 81 and 82. Alternatively, only pulp fibers exclusively could be conveyed to and through the blending systems 81 and 82 and the forming heads 71 and 72, respectively, when superabsorbent material and synthetic polymer fibers are discharged from the forming heads 91 and 92, respectively.

The material from each forming head is deposited, preferably with vacuum assist, as a loose, uncompacted, layer of material with the first layer lying directly on the tissue web or carrier layer 62 (or directly onto the endless screen 60).

The absorbent material may include a top carrier layer or cover layer, such as the cover layer 38 in the embodiment 20′ described above with reference to FIG. 2. If such a covered, absorbent material is to be produced, then a cover layer sheet or web 96 is unwound from a cover layer web roll 98 downstream of the forming heads and is directed over the previously deposited material as illustrated in FIG. 3.

The absorbent material is conveyed, preferably with the help of a conventional vacuum transfer device 100, from the end of the endless screen 60 to an embossing station comprising an upper roll 121 and a lower roll 122 which compresses or compacts the material to form an increased density web.

In the preferred contemplated embodiment, the upper roll 121 is typically a steel roll, and the lower roll 122 is typically a flexroll having a hardness of about 85 SH D. In the preferred process, the upper roll 121 has an embossing pattern surface, and the lower roll 122 has a smooth surface. In some applications it may be desirable to reverse the orientation of the web through the rolls so that the embossing roll contacts the carrier layer 62 of the web. In other applications, it may be desirable to provide both the upper and lower rolls 121 and 122 with an embossing pattern surface.

The weight of the upper roll 121 bears on the web. Additional force may be provided with conventional hydraulic actuators (not illustrated) acting on the axle of the roll 121. In one form of the invention, the web is compacted between the rolls 121 and 122 under a load of between about 28 and about 400 newtons per millimeter of transverse web width (160-2284 pounds force per inch of transverse web width).

The processing line is preferably run at a line speed of between about 30 meters per minute and about 300 meters per minute. Each roll 121 and 122 is heated, in the preferred embodiment, to at least about 120° C. The temperature of the rolls 121 and 122 should be sufficient to facilitate the establishment of hydrogen bonding of the pulp fibers to each other, as well as of the tissue layer (if any) to the pulp fibers, so as to increase the strength and integrity of the finished absorbent material. This provides a finished product with exceptional strength and resistance to shake-out of superabsorbent material.

The temperature of each roll is dependent upon the line speed and type of synthetic polymer fiber that is employed. It has been found that the process of the present invention can be operated to provide an absorbent material which, while having improved fluid acquisition properties imparted by the synthetic fibers, still has a relatively low Gurley Stiffness and is therefore soft and supple. To this end, according to one preferred form of invention, the process maintains the temperatures of the rolls 121 and 122 at temperatures which are sufficient to form liquid-stable bonds between the synthetic polymer fibers and the cellulosic fibers. The term “liquid-stable bonds” refers to bonds which do not significantly degrade over time when subjected to typical fluids with which the absorbent material is intended to be used (e.g., human body fluids).

According to one preferred form of the invention, the temperatures of the rolls 121 and 122 are not sufficient to cause melting of too much of the surface area of the synthetic polymer fibers incorporated in the web at the particular line speed and compaction load that are employed. Preferably, no more than about one half of the surface area of exteriors of the synthetic polymer fibers is melted. More preferably, significantly less surface area is melted. By avoiding significant melting of the surfaces of the synthetic polymer fibers, the process minimizes the formation of resolidified thermal bonds that would increase rigidity and stiffness of the web.

According to one aspect of a preferred form of the invention, the rolls 121 and 122 are provided with an embossing pattern, examples of which are described in detail hereinafter. Such an embossing pattern provides limited areas of greater compaction and adjacent areas of much lesser compaction. The areas of the web which are compacted more greatly by raised portions of the embossing pattern of the rolls are subjected to greater heat transfer and pressure from the rolls, and this can effect a melting of surface portions of the synthetic polymer fibers, followed by subsequent resolidification, to create thermal bonding to adjacent cellulosic fibers as well as adjacent synthetic polymer fibers. However, in the regions of the web which lie between the raised portions of the embossing pattern, little or no thermal bonding occurs between the synthetic polymer fibers and the adjacent cellulosic fibers. By providing a relatively large proportion of such unbonded or minimally bonded regions throughout the entire web, the stiffness of the resulting web can be controlled so that it remains relatively soft and supple. On the other hand, owing to the embossing pattern of raised portions adjacent which significant thermal bonding occurs between the synthetic polymer fibers and the cellulosic fibers, sufficient rigidity is imparted to the web along with sufficient fluid absorption capacity so as to provide a web which still has good fluid acquisition and absorptive capabilities as well as good strength and integrity.

Upon leaving the rolls 121 and 122, the web contains very little moisture (e.g., 1%-8% moisture based on the total weight of the web). The compressed and densified web is wound into a roll 130 using conventional winding equipment. The web moisture content will typically increase as the web reaches equilibrium with the ambient atmosphere, but it is desirable that the moisture content not be too high—preferably be between about 1% and about 8% of the total weight of the web.

The high density absorbent material made by the process of the present invention that contains superabsorbent material and synthetic polymer fibers has good fluid acquisition and absorptive capabilities, is surprisingly and unexpectedly soft and supple, and yet is relatively strong with good integrity, both wet and dry. The absorbent material can be prepared by the process of the present invention over a wide range of basis weights without adversely affecting its softness or strength.

The unique combination of strength, absorptive capability, and suppleness of absorbent material of the present invention which can be made by the process of the present invention has significant advantages to a manufacturer of absorbent articles. Typically, such a manufacturer purchases pulp, and then processes that pulp on-line in a manufacturing plant as the final article (e.g., diaper, sanitary napkin) is being made. Such processing steps may include defibering of the pulp, adding superabsorbent and the like. In an on-line system, the rapidity with which such steps can be carried out is limited by the slowest of the various steps. An example of a system that requires such processing steps (e.g., defibering) is disclosed in U.S. Pat. No. 5,262,005.

The need of the manufacturer to defiberize or otherwise process existing materials on-line means that the overall production process is substantially more complex. Further, the manufacturer must purchase, maintain, and operate the equipment needed to carry out such processing steps. The overall production cost is thus increased.

The absorbent material of the present invention can be directly incorporated into a desired absorbent article without the need for such processing steps. The manufacturer of the absorbent article does not have to defiber or otherwise treat the absorbent material made by the process of the present invention in any way other than shaping the absorbent material into the desired shape. In this way, the manufacturer can speed up the assembly process and realize substantial savings in cost and time.

A number of different forms of the absorbent material of the invention have been made according to the various forms of the process of the present invention. Samples of the various forms of the absorbent material were tested to evaluate various characteristics or properties. Characteristics or properties of the samples were also compared with characteristics of selected commercial products. The test results are set forth in Tables I, II, III, and IV and are discussed in detail following a description of the various test procedures and measurements set forth immediately below.

Measurements and Test Procedures

Basis Weight Determination

The basis weight of an absorbent material is determined from a specimen of the material by first weighing the specimen. The length and width of the specimen is the measured. The length and width are multiplied to calculate the area. The weight is then divided by the area, and the quotient is the basis weight.

Density Determination

The density of an absorbent material is determined from a specimen of the material by first weighing the specimen. The length, width, and thickness are measured and multiplied together to calculate the volume. The specimen weight is then divided by the volume to calculate the density.

Gurley Stiffness Determination

The “Gurley Stiffness” of an absorbent material is determined from a specimen of the material which is tested according to the conventional Gurley Stiffness test used in the nonwoven, absorbent fiber art. The Gurley Stiffness values of the absorbent material are measured using a Gurley Stiffness Tester (Model No. 4171E), manufactured by Gurley Precision Instruments of Troy, N.Y., U.S.A. The instrument measures the externally applied moment required to produce a given deflection of a test specimen strip of specific dimensions fixed at one end and having a concentrated load applied to the other end. The results are obtained in “Gurley Stiffness” values in units of milligrams. The greater the value of Gurley Stiffness of the material, the less flexible, and hence, the less soft, it is.

The inverse of Gurley Stiffness divided by 1000, expressed in units of inverse grams (g⁻¹), is defined as the “suppleness,” and is thus a measure of the softness, bendability and flexibility of an absorbent material.

Wicking Energy and Normalized

Wicking Energy Determination

Wicking is the ability of an absorbent material to direct fluid away from the point of fluid entry and distribute that fluid throughout the material.

The wicking capability of an absorbent material can be better characterized by expressing the wicking properties over the entire length of a tested sample. By calculating the total amount of fluid absorbed and wicked by a test sample (calculating the areas under a plot of absorbed fluid vs distance), a wicking energy (the capacity of the absorbent material to perform absorptive work) can be calculated.

Because absorption is in part a function of superabsorbent material content, that energy can be normalized for superabsorbent material content. The resulting value is referred to herein as “normalized wicking energy” and has the units ergs/g.

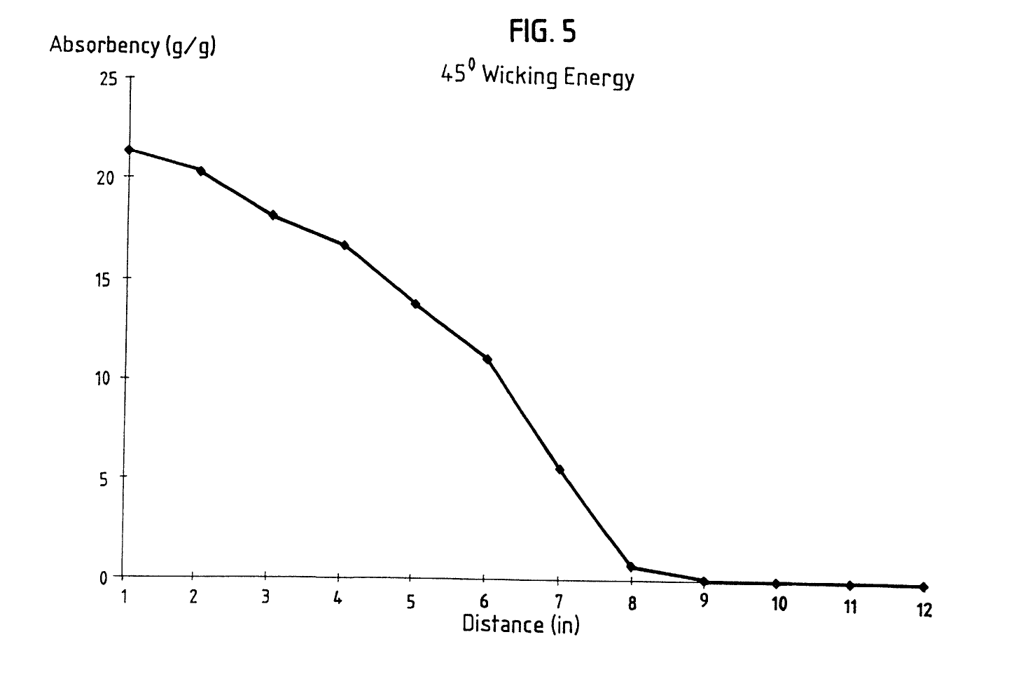

FIG. 4 illustrates the set up of the wicking test. A 45° wicking test cell is attached to the absorption measurement device. The test cell essentially consists of a circular fluid supply unit for the test sample and 45° ramps. The fluid supply unit has a rectangular trough and liquid level is maintained at the constant height by the measuring unit. A test sample having dimension of 1″×12″ is prepared. The sample is marked every inch along the length of the sample. The sample is then placed on the ramp of the test cell ensuring that one of the edges of the sample dips into the trough. The test is conducted for thirty minutes. The sample is removed after the specified period and cut along the marked distances. The cut pieces are placed into pre-weighed aluminum weighing dishes. Each weighing dish containing a wet sample is weighed again and then oven dried to a constant weight. By conducting a proper mass balance on the data, absorbency of the sample is determined at every inch. For each sample, the amount of fluid absorbed per gram of sample is plotted against distance from the origin (source of fluid). A representative plot is shown in FIG. 5. The area under the curve is calculated using the following formula:

[(y₁)(x₂−x₁)+0.5 (y₂−y₁)(x₂−x₁)+(y₂)(x₃−x₂)+0.5(y₃−y₂)(x₃−x₂)+ . . . +(yₙ)(xₙ−xₙ₋₁)+0.5(yₙ−yₙ₋₁)(xₙ−xₙ₋₁)],

where Xᵢ is distance at the i^{th} inch an Yᵢ is absorbency at the i^{th} inch.

This area was then multiplied by the gravitational constant (981 cm/s²) and the sine of 45° to result in the work value or wicking energy value expressed in units of ergs/g. The derived energy value is normalized for the varying superabsorbent material content by dividing by the percent superabsorbent material (% SAP) content.

Fluid Acquisition and Rewet Determination

Samples can be tested for (1) fluid acquisition, and (2) rewet using standard procedures well known in the art. These tests measure the rate of absorption of multiple fluid insults to an absorbent product or material and the amount of fluid that is rewet under 0.5 psi load. This method is suitable for all types of absorbent material, especially those intended for urine application.

The fluid acquisition and rewet test initially records the dry weight of a 40 cm by 12 cm (or other desired size) test specimen of the absorbent product or material. Then, an 80 milliliter, fixed volume amount or of saline solution is applied to the test specimen through a fluid delivery column at a 1 inch diameter impact zone under a 0.1 psi load. The time (in seconds) for the entire 80 milliliters of solution to be absorbed is recorded as the “acquisition time,” and then the test specimen is left undisturbed for a 30 minute waiting period. A previously weighed filter paper (e.g., Whatman #4 (70 mm)) is placed over the solution impact zone, and a 0.5 psi load is then placed on the filter on the test sample for 2 minutes. The wet filter paper is then removed, and the wet weight is recorded. The difference between the initial dry filter weight and final wet filter weight is recorded as the “rewet value” of the test specimen. This entire test is repeated 2 times on the same wet test specimen. Each acquisition time and rewet volume is reported along with the average and the standard deviation. The “acquisition rate” is determined by dividing the 80 milliliter volume of liquid used by the acquisition time previously recorded. For any specimen having one embossed side, the embossed side is the side initially subjected to the test fluid.

Test Sample Production Processes and Examples

### EXAMPLE 1

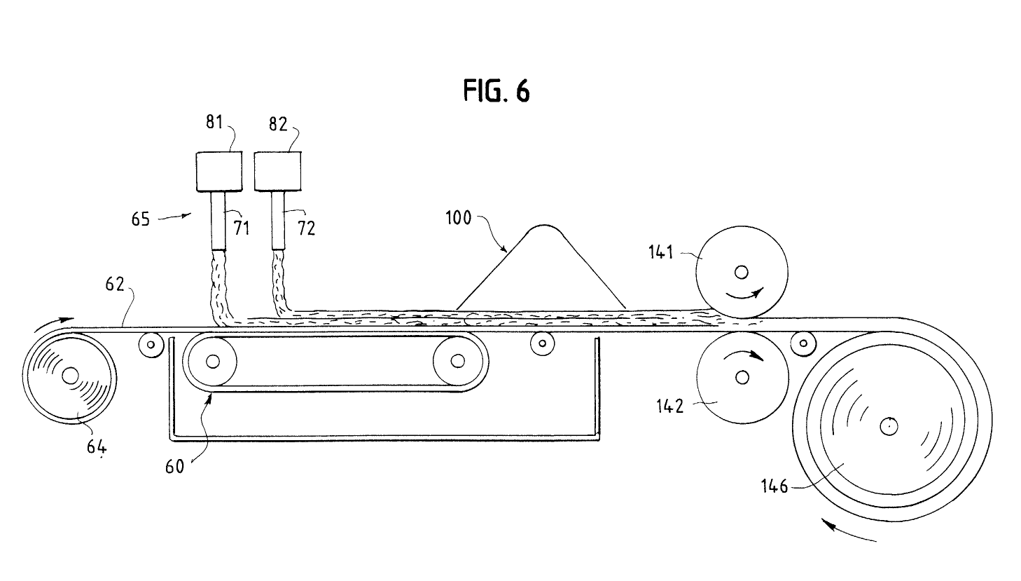

In Example 1, the form of the present invention illustrated in FIG. 1 was made by the process generally illustrated in FIGS. 6-8 with a variety of different synthetic polymer fiber compositions listed in Table I.

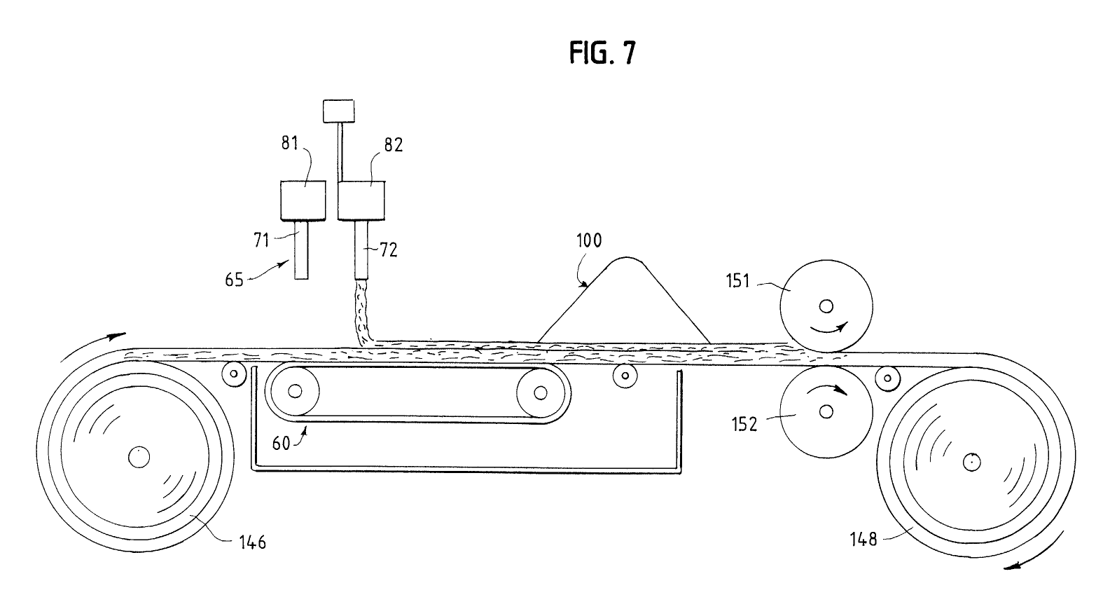

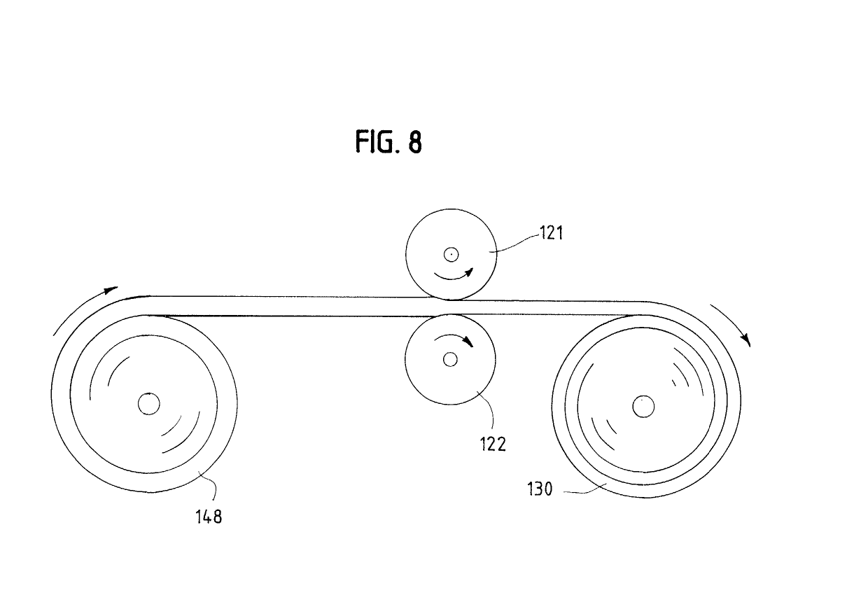

The various specimen rolls of absorbent material were made by initially partially forming the web of material in a first stage on the apparatus shown in FIG. 6, then subsequently running the partially completed web again in a second stage of the process on the apparatus as shown in FIG. 7. Subsequently, the second stage web was embossed in a third stage on a processing line as shown in FIG. 8. The processing line shown in FIGS. 6 and 7 is generally similar to the above-described preferred processing line illustrated in FIG. 3. The processing line shown in FIGS. 6 and 7 includes the carrier web roll 64 from which is drawn a carrier web 62 over an endless screen 60 located under a series of forming heads 65, including a first forming head 71 and a second forming head 72 which are connected with blending systems 81 and 82, respectively.

At the end of the endless screen 60 is a conventional vacuum transfer device 100, and downstream of the vacuum transfer device 100 is a compaction station comprising an upper roll 141 and a lower roll 142. These are conventional, smooth-surfaced compaction rolls heated to about 60° C. Downstream of the compaction rolls 141 and 142, the partially completed, first stage web is wound onto a first intermediate roll 146.

In the first stage of the process illustrated in FIG. 6, the first forming head 71 deposited pulp fibers and superabsorbent particles on the carrier layer 62, and the second forming head 72 deposited only pulp fibers.

In all of the specimens made according to this process as listed in Table I, the same material was used for carrier layer 62. It was a tissue sold by Cellu Tissue Corporation, 2 Forbes Street, East Hartford, Conn. 06108, U.S.A. under the designated grade 3008. It is produced from 100% southern softwood and had a basis weight of 10-11 pounds per 3000 square feet. It had a dry tensile strength in the machine direction of 250-275 grams per inch and a dry tensile strength in the cross direction of 50-60 grams per inch. The elongation in the machine direction at the breaking point was 22-28%. The porosity was 285 cubic feet per minute per square foot. The brightness reflectance was 78 at 457 mm.

In the first stage of the forming process of Example 1 illustrated in FIG. 6, the pulp fibers deposited from the first forming head 71 (along with the superabsorbent particles) and pulp fibers deposited from the second forming head 72 were untreated pulp fibers identified as Rayfloc-J-LD fibers made by Rayonier, Inc. having an office at 4474 Savannah Highway, Jesup, Ga. 31545, U.S.A. The base web material produced in the first stage by the process illustrated in FIG. 6 had a basis weight of 500 grams per square meter and contained 55% by weight of superabsorbent particles.

The superabsorbent material used in the first stage of the process was deposited along with the pulp fibers from the first forming head 71 in the form of superabsorbent particles sold under the designation SXM 7440 by Stockhausen GmbH, Krefeld, Germany, and having an office at 2401 Doyle Street, Greensboro, N.C. 27406, U.S.A.

The first stage base web was lightly compacted in the by the compaction rollers 141 and 142 solely for purposes of establishing some minimum amount of handling integrity, after which the web was wound onto the roll 146.

In the second stage of Example 1, as illustrated in FIG. 7, the web 146 (produced by the first stage of the process shown in FIG. 6) was installed at the beginning of the process line and run through the process line while additional material was deposited thereon from the forming head 72. In the second stage of the process, the forming head 72 deposited a mixture or blend of pulp fibers and synthetic polymer fibers. Forming head 71 was not operated. The blend of synthetic polymer fibers and pulp deposited from the forming head 72 in the second stage of the process illustrated in FIG. 7 was an equal weight blend of 50% pulp fibers and 50% synthetic polymer fibers. The pulp fibers deposited in the second stage were the same type as used in the first stage of the process illustrated in FIG. 6 and described above. Three different types of synthetic polymer fibers were separately used as listed in Table I to produce various specimens.

All of the types of synthetic polymer fibers were provided in a conventional high-crimped (“HC”) condition in which the fibers are twisted and curled. In Table I, in the first column identifies two polymer fibers: “PP,” which designates polypropylene, and “PET,” which designates polyethylene terephthalate. Each synthetic fiber specimen listed in the first, left-hand end column of Table I includes the “dtex” number which designates the weight in grams of 10,000 meters of such a fiber. Also listed in the left-hand end column of Table I for each synthetic polymer fiber type is the nominal length of the fibers in millimeters (mm).

In the second stage of the process illustrated in FIG. 7, the further completed web, which then contained the synthetic polymer fibers, was run between a pair of calender rolls 151 and 152 while the compaction rolls 141 and 142 (illustrated in FIG. 6 but omitted from FIG. 7) were disengaged from the line. The calender rolls 151 and 152 were smooth surfaced rolls maintained at a temperature of 140° C. The calendered web was then wound onto a roll 148.

The Example 1 web as produced in the second stage illustrated in FIG. 7 had a total basis weight of 550 grams per square meter with a superabsorbent polymer content of about 50% by weight and a total fiber content (both pulp fiber and synthetic polymer fiber) of about 50% by weight. The amount of pulp fibers in the mixture of fibers was about 91% by weight, and the amount of synthetic polymer fibers in the mixture of fibers was about 9% by weight.

As illustrated in FIG. 8, in the third stage of the process for producing the Example 1 specimens listed in Table I, the calendered roll 148 from the second stage was run through an embossing station comprising an upper embossing roll 121 and a lower roll 122 which were of the type described above with respect to the preferred embodiment of the process illustrated in FIG. 3. In particular, the lower roll 122 was a smooth-surfaced roll, and the upper roll 121 contained an embossing pattern in its surface.

Each of the rolls 121 and 122 was maintained at an elevated temperature of 151° C. The rolls 121 and 122 were maintained to provide a compaction load on the web of about 240 pounds per linear inch of transverse web width. The embossed web was wound on a roll 130 (FIG. 8).

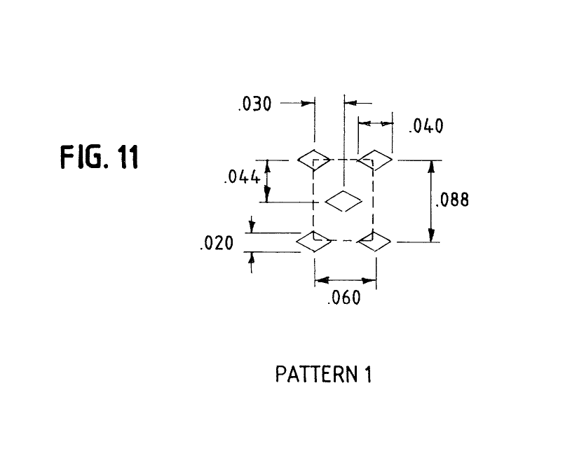

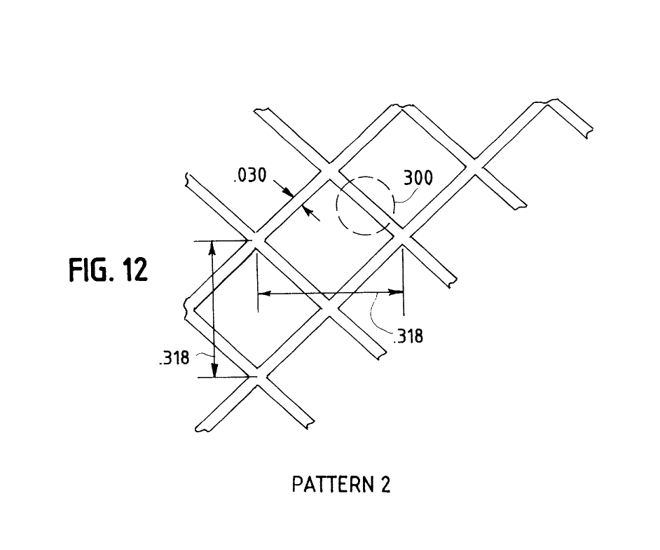

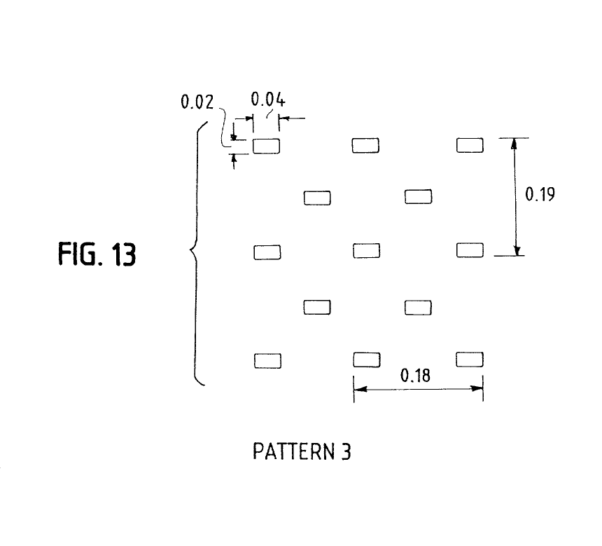

Three different embossing rolls 121 were separately employed on different specimen runs to provide different embossing patterns on various specimens. The embossing rolls 121 had surface indentations so as to create a pattern of depressions and raised areas (relative to the depressions). The basic repeating unit of each of the three embossing patterns are illustrated in FIGS. 11, 12, and 13, respectively. In the second column of Table I, each of the three embossing patterns is designated with the unique identifier number: 1 (FIG. 11), 2 (FIG. 12), or 3 (FIG. 13), respectively. The pattern repeating unit dimensions are indicated in the Figures in units of inches. The depth of the embossing roll surface depressions is 0.03 inch for pattern 1, 0.03 inch for pattern 2, and 0.03 inch for pattern 3. The raised area surface is 15% of the total roll pattern area for pattern 1, 25% of the total roll pattern area for pattern 2, and 10.8% of the total roll pattern area for pattern 3. For pattern 3, the number of raised surface areas per square inch is 142.

In Table I, specimens were made with one of three different types of synthetic polymer fibers: (1) PP-6.7 dtex, 6.0 mm, HC; (2) PET-6 denier, 6.35 mm, HC; and (3) PET-17 denier, 12.7 mm, HC. The polypropylene (“PP”) fibers and polyester (“PET”) fibers were provided by Mini Fiber, Inc., 2923 Boones Creek Road, Johnson City, Tenn. 37615 U.S.A. A control specimen was made without adding any synthetic polymer fibers, and this is listed in the first row of Table I as “Control Sample.” Each of the three identical samples each of the three types of synthetic polymer fiber specimens was embossed with a different one of the three types of embossing patterns as indicated in the second column of Table I. A fourth sample of each of the three types of synthetic polymer fiber specimens was taken from a web which was not embossed (i.e., the specimens were taken from the roll 148 at the end of the second stage (FIG. 7). The Control Sample was also taken from a specimen at the end of the second stage (FIG. 7) so that the Control Sample was not embossed.

In Table I, the values for the density, stiffness, fluid acquisition, and rewet are listed as the average of measurements or tests of six individual test samples.

In Table I, the columns “Acq 1,” “Acq 2,” and “Acq 3” designate the fluid acquisition rate as determined by the fluid acquisition test procedure described in detail above.

In Table I, the columns designated “Rwet 1,” “Rwet 2,” and “Rwet 3” designate the rewet values determined from the rewet test described above in detail.

Table I also lists density, stiffness, and suppleness values for three different commercial products which have a basis weight that is generally comparable to the basis weight of the materials of the present invention which were tested to provide the results listed in Table I. However, the three commercial products have substantially lower densities than the material of the resent invention.

The commercial product designated “Concert 500.384” is a thermally bonded, air-laid, absorbent core sold by Concert Fabrication Ltee, having an office at Thurso, Quebec, Canada, and has a total basis weight of 500 grams per square meter and is comprised of fluff pulp at 240 grams per square meter, bonding fiber at 35 grams per square meter, and superabsorbent material at 225 grams per square meter. The thickness is 4.20 millimeters, the density is 0.12 grams per cubic centimeter, the dry tensile strength in the machine direction is 1100 grams per 50 millimeters, the absorbent capacity is 32 grams per gram water at 2 minutes, and 18 grams per gram saline at 2 minutes. The brightness is 86%, and the rewet is 1.4 grams per insult after the third insult of 5 millileters, 1.8 grams per insult after the fourth insult at 5 millileters, and 0.5 grams per insult after the fifth insult at 5 millileters. The saline spread/wicking rate is 50 millileters diameter/S millileters/2 minutes.

The commercial product designated in Table I as “Concert 500.382” is also a thermally bonded, air-laid, absorbent core sold by Concern Fabrication Ltee. The commercial product designated “Concert 500.382” has a total basis weight of 500 grams per square meter and is comprised of fluff pulp at 215 grams per square meter, bonding fiber at 35 grams per square meter, and superabsorbent material at 250 grams per square meter. The thickness is 4.20 millimeters, the density is 0.12 grams per cubic centimeter, the dry tensile strength in the machine direction is 1100 grams per 50 millimeters, the absorbent capacity is 32 grams per gram water at 2 minutes, and 18 grams per gram saline at 2 minutes. The brightness is 85%, and the rewet is 0.8 grams per insult after the second insult of 5 millileters and is 1.2 grams per insult after the third insult at 5 millileters. The saline spread/wicking rate is 50 millileters diameter/5 millileters/2 minutes.

The commercial product designated in Table I as “Merfin 44500T40” is provided by Merfin International, Inc. having an office at 7979 Vantage Way, Della, British Colombia, Canada V4G186. The Merfin product is a thermally bonded, air-laid, absorbent core having a total basis weight of 450 grams per square meter, a superabsorbent material content with a basis weight of 183 grams per square meter, a thickness of 2.95 millimeters per ply, a density of 0.156 grams per cubic centimeter, a dry tensile strength in the machine direction of 1100 grams per 25.4 millimeters, and a dry tensile strength in the cross direction of 850 grams per 25.4 millimeters. The product had an absorbency of 15.7 grams per gram for 0.9% saline solution and a retention of 84.6%. The rewet for a 0.9% saline solution 50 milliliter insult of 0.9% saline solution is 0.1 grams after the first insult, 5.7 grams after the second insult, and 14.3 grams after the third insult.

Upon consideration of the values listed in Table I, the absorbent material of the present invention produced by the three-stage process described above with reference to FIGS. 6-8 is seen to have substantially lower stiffness (and therefore greater suppleness) when compared to commercial air-laid products having a comparable basis weight, even though such products have a substantially lower density than the material of the present invention.

TABLE I

| | Emboss | | | | | | | | | |
|---|---|---|---|---|---|---|---|---|---|---|
| Synthetic Polymer Fiber Type | ing | Density | Stiffness | Suppleness | Acq 1 | Acq 2 | Acq 3 | Rwet 1 | Rwet 2 | Rwet 3 |
| Sample Run Identification | Pattern | (g/cc) | (mg) | (1/g) | (ml/s) | (ml/s) | (ml/s) | (g) | (g) | (g) |
| Control Sample--No synthetic | None | 0.35 | 1212 | 0.83 | 1.43 | 1.52 | 1.29 | .05 | .05 | 8.40 |
| fibers used | | | | | | | | | | |
| PP-6.7 dtex, 6.0 mm, HC | None | 0.30 | 648 | 1.54 | 1.35 | 2.47 | 1.92 | 0.05 | 2.19 | 9.87 |
| PP-6.7 dtex, 6.0 mm, HC | 3 | 0.34 | 750 | 1.33 | 1.35 | 2.14 | 1.73 | 0.06 | 0.10 | 22.91 |
| PP-6.7 dtex, 6.0 mm, HC | 2 | 0.32 | 855 | 1.17 | 1.19 | 1.83 | 1.66 | 0.07 | 0.20 | 10.54 |
| PP-6.7 dtex, 6.0 mm, HC | 1 | 0.30 | 983 | 1.02 | 3.35 | 3.60 | 2.98 | 0.05 | 0.58 | 21.48 |
| PET-6 denier, 6.35 mm, HC | None | 0.32 | 736 | 1.36 | 1.36 | 2.27 | 1.79 | 0.03 | 1.90 | 11.37 |
| PET-6 denier, 6.35 mm, HC | 3 | 0.32 | 728 | 1.37 | 1.34 | 1.89 | 1.47 | 0.07 | 0.88 | 21.06 |
| PET-6 denier, 6.35 mm, HC | 2 | 0.34 | 770 | 1.30 | 1.21 | 2.01 | 1.72 | 0.06 | 1.48 | 21.54 |
| PET-6 denier, 6.35 mm, HC | 1 | 0.32 | 876 | 1.14 | 1.43 | 2.26 | 1.83 | 0.36 | 1.98 | 22.17 |
| PET-17 denier, 12.7 mm, HC | None | 0.33 | 760 | 1.32 | 1.40 | 2.17 | 1.82 | 0.10 | 0.40 | 17.18 |
| PET-17 denier, 12.7 mm, HC | 3 | 0.32 | 675 | 1.48 | 1.59 | 2.31 | 2.09 | 0.17 | 0.21 | 16.57 |
| PET-17 denier, 12.7 mm, HC | 2 | 0.35 | 803 | 1.25 | 1.46 | 2.32 | 2.03 | 0.05 | 0.13 | 21.51 |
| PET-17 denier, 12.7 mm, HC | 1 | 0.31 | 750 | 1.33 | 2.08 | 2.89 | 2.49 | 0.07 | 0.26 | 21.95 |
| Concert 500.384 (45% SAP) | | 0.12 | 1640 | 0.61 | | | | | | |
| Concert 500.382 (50% SAP) | | 0.12 | 1535 | 0.65 | | | | | | |
| Merfin 44500T40 (40% SAP) | | 0.17 | 2702 | 0.37 | | | | | | |

### EXAMPLE 2

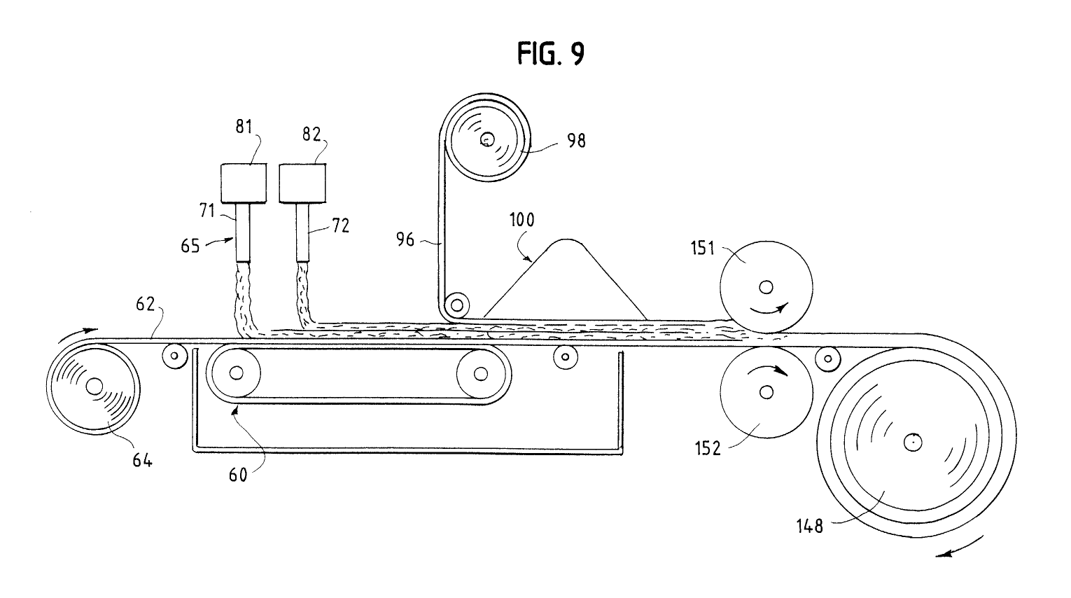

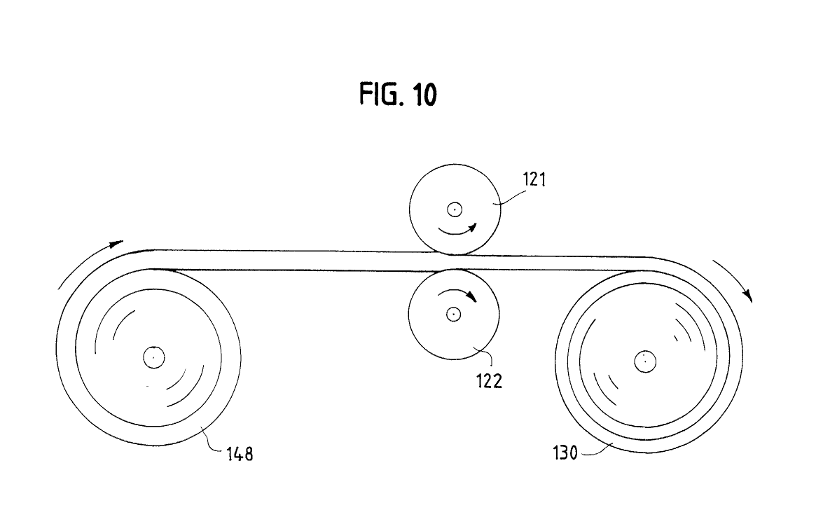

In Example 2, specimens of the present invention having a configuration illustrated in FIG. 2 were made and evaluated. The structure in FIG. 2 includes a cover layer 38 in addition to the carrier layer 22 attached to the primary absorbent portion or core 36. The specimens were produced according to the two-stage process illustrated in FIGS. 9 and 10. Material formed by the first stage of the process illustrated in FIG. 9 is wound on a roll 148 and is used as the beginning roll in the second stage of the process illustrated in FIG. 10.

The first stage of the process illustrated in FIG. 9 is similar in many respects to the preferred process described above with reference to FIG. 3. In particular, in the first stage of the process illustrated in FIG. 9, the carrier layer web 62 is unwound from a roll 64 and is directed onto an endless screen 60 over which is forming station 65 having a forming head 71 and forming head 72 each connected with a blending system 81 and 82, respectively. The cover layer 38 is initially provided in the form of a cover layer web 96 unwound from a roll 98.

Cellulosic fibers or pulp fibers were discharged from the first forming head 71 along with superabsorbent particles onto the carrier layer 62. Pulp fibers together with synthetic polymer fibers were discharged from the second forming head 72.

The pulp fibers used in both forming heads 71 and 72 were a blend of (1) the Rayfloc J-LD pulp described above with reference to Example 1, and (2) cold caustic treated fibers as defined above with reference to U.S. patent application Ser. No. 08/370,571 filed Jan. 18, 1995 and as described above. In Table II, in the second column from the left-hand end (entitled “Absorbent Core Fiber Blend”), the Rayfloc-J-LD pulp is designated with the upper case letter “A,” and the cold caustic treated pulp is designated with the upper case letter “B.” The percentage of each type of pulp is listed in Table II on a weight basis with respect to the total weight of the absorbent core portion—but not including the carrier layer and cover layer (carrier layer web 62 in FIG. 9 or layer 22 in FIG. 2, and cover layer web 96 in FIG. 9 or cover layer 38 in FIG. 2).

In Table II, the second column from the left-hand end (entitled “Absorbent Core Fiber Blend”) lists the synthetic polymer fibers under the designation “Bico,” and this identifies bicomponent fibers which are provided by FiberVisions Company, having an office at Engdraget 22, DK-6800 Varde, Denmark. The particular bicomponent fiber is sold under the designation “AL Adhesion” and is a 1.7 dtex fiber having a nominal length of 6 mm with a central core that is 50% by weight polypropylene surrounded by a sheath that is 50% by weight polyethylene.

In Table II, a Control Run sample is listed in the last row and was made by the process illustrated in FIGS. 9 and 10, except that no synthetic polymer fibers were blended with the pulp fibers in either the forming head 71 or the forming head 72 for the Control Run.

In the forming head 71, the pulp fibers were blended with superabsorbent particles of the same type as used in Example 1, namely the Stockhausen product designated as SXM 4750. The absorbent core portion between the carrier layer web 62 and cover layer web 96 for each sample run had a basis weight of 400 grams per square meter, and the superabsorbent articles 40 were 40% of the basis weight of the core portion. The remaining 60% of the basis weight was made up of the regular pulp fibers “A,” the cold caustic treated pulp fibers “B,” and the bicomponent synthetic polymer fibers (“Bico”) according to the percentages listed in the Table II second column from the left-hand end (entitled “Absorbent Core Fiber Blend”).

The carrier layer 62 is identified in the Table II third column from the left-hand end (entitled “Carrier Layer”) as either “Tissue” or “Pantex.” The tissue was the same type of tissue that was used in Example 1 described above. The term “Pantex” designates an through air bonded carrier layer or sheet 62 sold under the designation AB/S 22 by Pantex sr1 having an office at Via Terracini, snc, Loc. Spedalino Asnelli I-51031 Agliana (PT)-Italy. The Pantex carrier sheet had a basis weight of 22 grams per square meter, a thickness of 430 micrometers (+ or 15%), a tensile strength per European Disposables And Nonwovens Association (“EDANA”) standard 20.2-89 of at least 5 N/50 mm in the machine direction at least 1 N/50 mm in the cross direction, an elongation per EDANA standard 20.2-89 of not more than 35% in the machine direction and not more than 55% in the cross direction, and a strike through time per EDANA standard 150.2-93 of not more than 2 seconds.

The cover layer web 96 is identified in the Table II fourth column from the left-hand end (entitled “Top (Cover) Layer”). Sample Run C and the Control Run sample did not have a cover layer. Sample Run B and Sample Run A included a cover layer web 96 provided by Fibervisions Company (identified above) under the designation FiberVisions™ ES-C. That product had a basis weight of 40 grams per square meter and consisted of a 3.3 dtex bicomponent synthetic fiber polymer having a 50% by weight polypropylene core and a 50% by weight polyethylene sheath with a nominal fiber length of 40 mm.

With continued reference to FIG. 9, the absorbent core portion was carried along between the carrier layer web 62 and cover layer web 96 from the endless screen 60 with the help of a conventional vacuum transfer device 100 to a calendering station comprising an upper roll 151 and a lower roll 152. The calendering rolls 151 and 152 each had a smooth surface, and each was maintained at a temperature of 140° C.

The web at the end of the first stage of the Example 2 process illustrated in FIG. 9 was wound onto an intermediate roll 148. Subsequently, the web roll 148 was transferred to a second processing station as illustrated in FIG. 10 where it was unwound and embossed at an embossing station comprising an upper roll 121 and a lower roll 122. The upper roll 121 had a surface embossing pattern corresponding to pattern 2 described above and illustrated in FIG. 11. The lower roll 122 had a smooth surface. The patterned top roll 121 and the smooth bottom roll 122 were maintained at temperatures for the various sample runs as identified in the Table II. The rolls 121 and 122 at the embossing station were maintained to provide the pressure or roll loading on the web as identified in the Table II column entitled “Pressure.” In that column, “psi” designates pounds per square inch and “PLI” designates pounds per linear inch of transverse web width. The embossed web was wound on a roll 130 (FIG. 10).

For Sample Run A, the lower carrier layer or “Pantex” side was embossed, but in the other three sample runs (Sample Run B, Sample Run C, and the Control Run), the embossing was applied to the top or cover layer. Thus, for the Sample Run A web, the web roll 148 had to be turned over at the embossing station (FIG. 10) compared to the orientation of the web roll 148 for the Control Run and other sample runs.

TABLE II

| | | | | Embossing Station | | | |
|---|---|---|---|---|---|---|---|
| | | | | Roll Temperature ° C. | | | |
| | Absorbent | | | Patterned | Smooth | | |
| Sample Run | Core Fiber | Carrier | Top (Cover) | Roll | Roll | Pressure | |
| Identification | Blend | Layer | Layer | (Top) | (Bottom) | (psi/PLI) | Comments |
| Sample Run C | 40% A, | Tissue | None | 142 | 123 | 25/150 | No top layer. |
| | 50% B, 10% | | | | | | Embossing pattern |
| | Bico | | | | | | on top side |
| Sample Run B | 38% A, | Pantex | FiberVisions | 148 | 123 | 23/138 | Embossing pattern |
| | 47% B, 15% | | | | | | on FiberVisions side |
| | Bico | | | | | | |
| Sample Run A | 38% A, | Pantex | FiberVisions | 124 | 125 | 23/138 | Embossing pattern |
| | 47% B, 15% | | | | | | on Pantex side. |
| | Bico | | | | | | |
| Control Run | 45% A, | Tissue | None | 110 | 110 | 12/75 | Control Sample--No |
| | 55% B | | | | | | synthetic fibers |

Table III lists the average of six measurements or tests performed on six sample specimens of each of the sample runs, and these measurements and tests are the same as described above with reference to Example 1. Table III also lists the density, stiffness, and suppleness values for two commercial products, and these commercial products are two of the same products described above with reference to Example 1.

It can be seen that the Table III Sample Runs A, B, and C of the present invention have a lower Gurley Stiffness (better or higher suppleness) than the commercial products which have lower densities.

TABLE III

| Sample Run | Embossing | Density | Stiffness | Suppleness | Acq 1 | Acq 2 | Acq 3 | Rwet 1 | Rwet 2 | Rwet 3 |
|---|---|---|---|---|---|---|---|---|---|---|
| Identification | Pattern | (g/cc) | (mg) | (1/g) | (ml/s) | (ml/s) | (ml/s) | g | g | g |
| Control | 012 | 0.37 | 991 | 1.10 | 0.64 | 0.70 | 0.55 | 0.08 | 0.13 | 13.20 |
| Sample Run A | 012 | 0.27 | 1130 | 0.88 | 1.09 | 1.44 | 1.25 | 0.06 | 10.55 | 21.47 |
| Sample Run B | 012 | 0.28 | 1183 | 0.85 | 1.11 | 1.25 | 1.07 | 0.07 | 11.57 | 19.16 |
| Sample Run C | 012 | 0.27 | 571 | 1.75 | 1.15 | 1.27 | 1.07 | 0.09 | 8.85 | 18.40 |
| Concert 500.382 | | 0.12 | 1640 | 0.61 | | | | | | |
| Merfin 44500T40 | | 0.17 | 2702 | 0.37 | | | | | | |

Table IV lists the density and percent superabsorbent for the various samples and also lists the value of the wicking energy and normalized wicking energy as determined by the wicking energy test described in detail above. The wicking energy test results are listed in Table IV for two commercial products which are two of the same commercial products described in detail above with respect to Example 1. From Table IV, it can be seen that the high density, soft, absorbent material of the present invention in Sample Run A, Sample Run B, and Sample Run C exhibits wicking capabilities which are comparable to those of the commercial, low density, air-laid, absorbent products.

TABLE IV

| | | | Wicking | Normalized Wicking |
|---|---|---|---|---|
| Sample Run | Density | SAP | Energy | Energy |
| Identification | (g/cc) | % | (Ergs/g) | (Ergs/g) |
| Control Run | 0.37 | 40 | 148679 | 3717 |
| Sample Run A | 0.27 | 40 | 73296 | 1832 |
| Sample Run B | 0.28 | 40 | 73296 | 1832 |
| Sample Run C | 0.27 | 40 | 78820 | 1971 |
| Concert 500.382 | 0.12 | 45 | 93,016 | 2067 |
| Merfin 44500T40 | 0.17 | 40 | 62094 | 1552 |

Additional Inventive Aspects

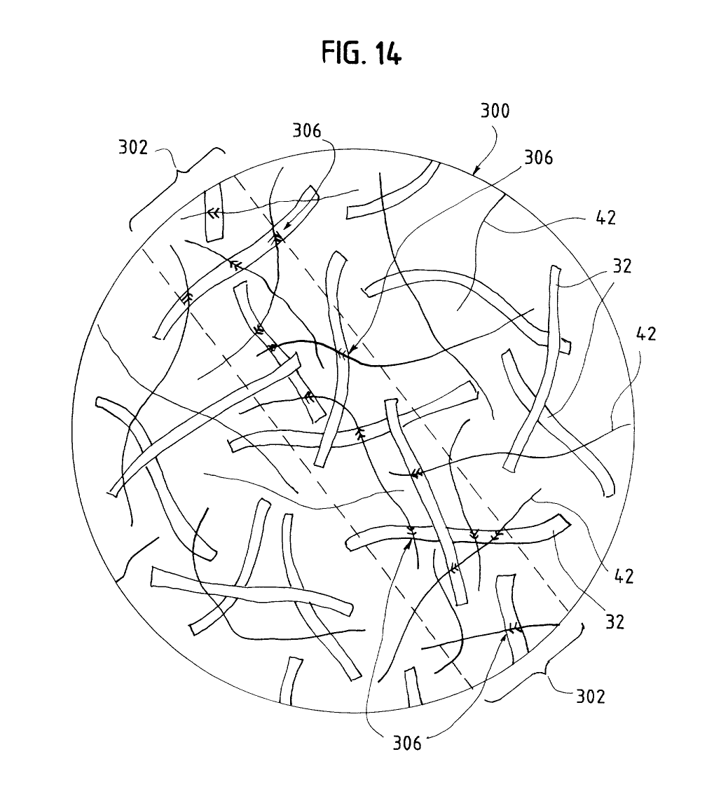

FIG. 14 diagrammatically illustrates an enlarged portion of a web of the present invention. The web is one that would have been produced according to the process used in Example 2 using the generally diamond-shaped embossing pattern No. 2 illustrated in FIG. 12. The region of the web illustrated in FIG. 14 would have been located within the circle designated 300 in FIG. 12 relative to the diamond-shaped embossing pattern of the upper embossing roll 121 (FIG. 8). As explained above, the FIG. 12 embossing pattern has a depth of 0.03 inch. That is, the narrow raised portions defining the diamond-shaped pattern have a height of 0.03 inch above the recessed interior portions. The surface area of the raised portions is only about 25% of the total roll pattern area. When the cellulosic fibers and synthetic polymer fibers comprising the web are moved under the embossing roll 121, portions of the web are contacted by the raised portions of the embossing pattern, and the adjacent portions of the web are received within the recessed portions of the pattern. The portions of the web in contact or registry with the raised portions of the pattern are compressed or compacted with greater force than the adjacent portions of the web. FIG. 14 illustrates the region in the completed web wherein the location of one of the embossing raised portions of the pattern would have been located as schematically represented by reference number 302 defined by two spaced-apart, parallel, dashed lines. In FIG. 14, the cellulosic pulp fibers are designated by the reference number 32, and the synthetic polymer fibers are designated by the reference number 42. In the region of the web that is in registry with the embossing pattern raised portion 302, there is significant bonding in the form of liquid-stable bonds between the cellulosic fibers 32 and the synthetic polymer fibers 42. In the preferred form of the present invention, such bonds are defined by thermal bonding of melted and subsequently resolidified portions of the synthetic polymer fibers which are in contact with the cellulosic fibers. Such bonding is effected in the greater compaction regions that are in registration with the embossing pattern raised portions 302. These thermal bonds are schematically represented by regions 306. The synthetic polymer fibers 32 are also bonded to each other within the region that is in registration with the embossing pattern raised portion 302.

In the areas of the web laterally on either side of the embossing pattern raised portions 302 there is little or no thermal bonding of the synthetic polymer fibers to the cellulosic fibers or of the synthetic polymer fibers to each other.

Because the raised portions 302 of the pattern No. 2 (FIG. 12) constitute only about 25% of the total pattern area of the embossing roll, the major portion of the total surface area of all of the synthetic polymer fibers is not melted and resolidified to form thermal bonds.

It should be understood that in the region of the web that is compacted in registry with the embossing pattern raised portion 302, there can be some synthetic polymer fibers that are near cellulosic fibers or other synthetic fibers but that are not bonded thereto. Similarly, in the regions of the web that are laterally displaced from the embossing pattern raised portion 302, there may be some bonding between a synthetic polymer fiber and a cellulosic fiber as well as between a synthetic polymer fiber and another synthetic polymer fiber. However, most of the bonding occurs in the relatively defined regions that are in registry with the embossing pattern raised portions 302.

As the web is compacted at the embossing station, the diamond-shaped arrangement of the embossing pattern raised portions 302 across the web results in the creation of substantial bonding in limited areas throughout the web. The pattern of limited bonding areas provides the web with strength and integrity without creating an undesirably stiff structure. Indeed, the web of absorbent material is relatively soft and supple. In a preferred form of the invention, at least a major portion of the total surface area defined on the exteriors of the synthetic polymer fibers has not been melted and resolidified, and the resulting web has a Gurley Stiffness of less than about 1500 mg, preferably less than about 1200 mg.

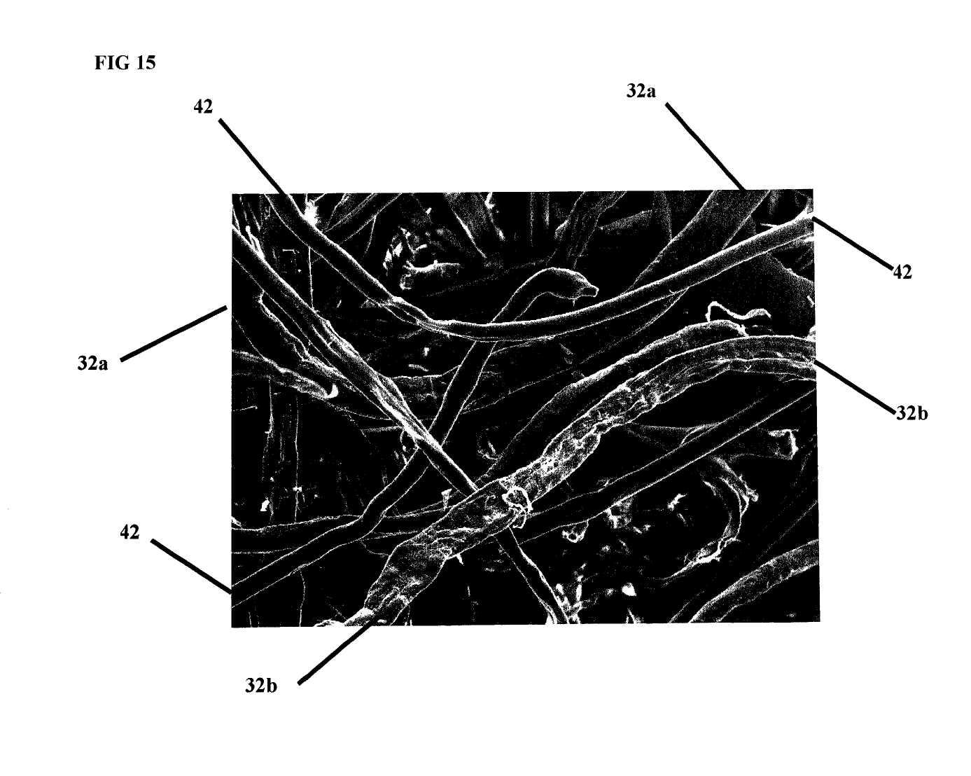

FIG. 15 is a scanning electron microscope photomicrograph of a sample of the material of the present invention showing a region of the web which was not in registry with the embossing pattern raised portions and hence, which was subjected to less pressure than regions that were in registry with the raised portions of the embossing pattern. FIG. 15 shows cellulosic fibers 32a and 32b and synthetic polymer fibers 42 in close proximity with very little or no bonding. The cellulosic fibers 32a are cold caustic treated fibers, and the cellulosic fibers 32b are not cold caustic treated.

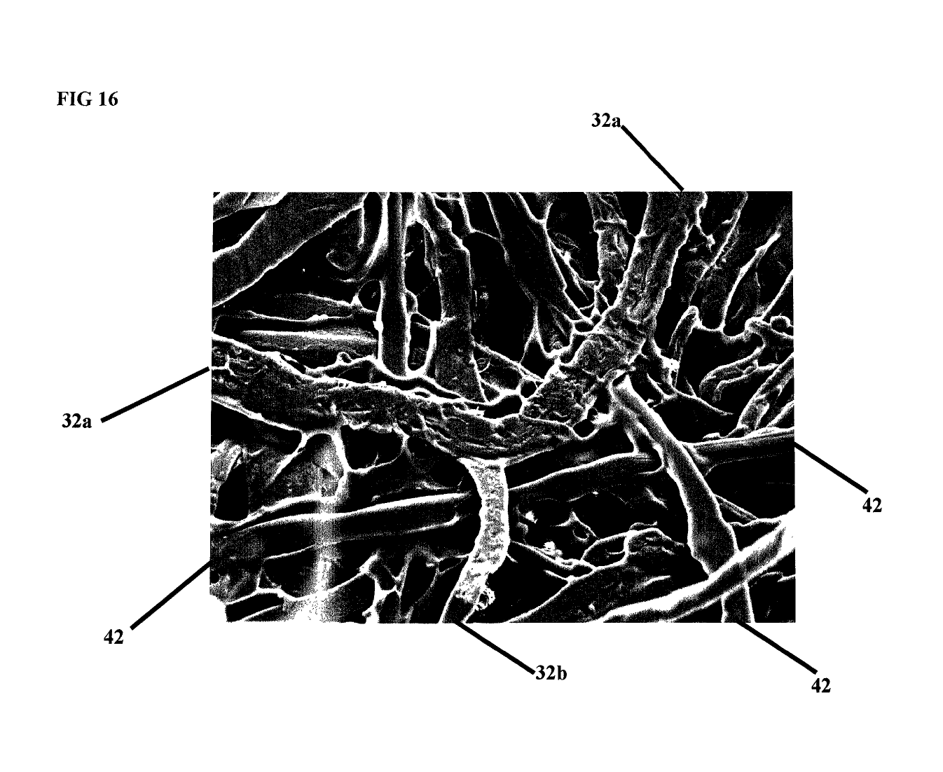

In contrast, FIG. 16 shows a region of the same material which was formed against or in registry with a raised portion of the embossing pattern (e.g., area 302 of the embossing pattern as schematically represented in FIG. 14). It can be seen in FIG. 16 that portions of the synthetic polymer fibers 42 have created thermal bonds with and between the cellulosic fibers 32a and 32b as well as with other synthetic polymer fibers 42.

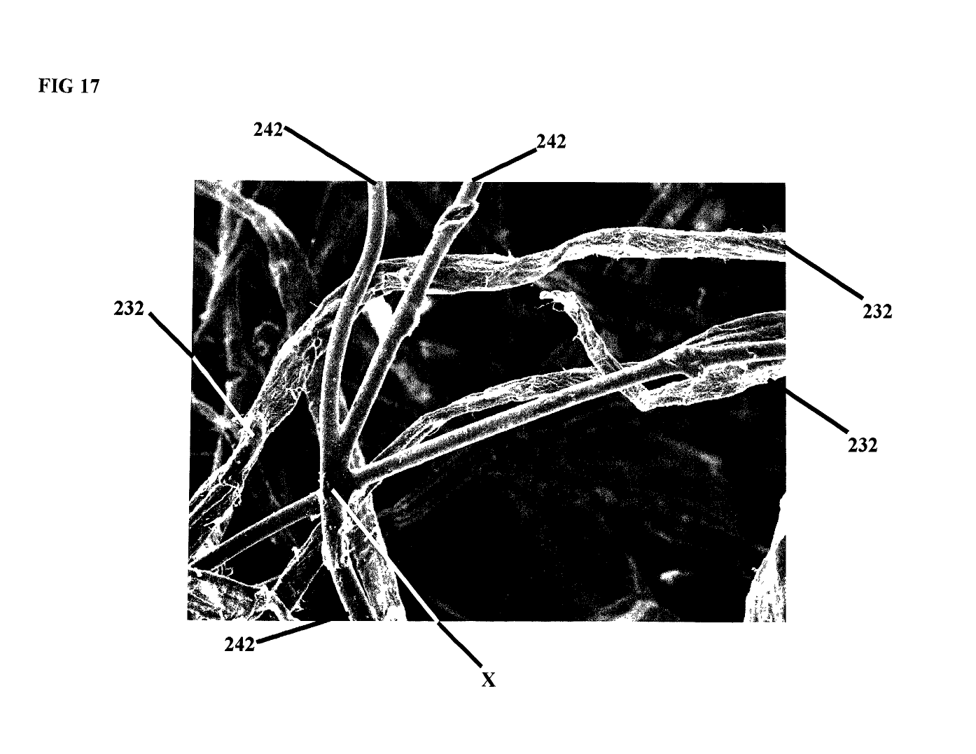

FIG. 17 is a scanning electron microscope photomicrograph of a portion of the commercial product identified in Table I as “Concert” 500.382 as described above. In FIG. 17, synthetic polymer fibers are identified by the reference number 242 and cellulosic fibers are designated by the reference number 232. In FIG. 17, it can be seen that two synthetic polymer fibers 242 cross each other at “X” and are thermally bonded. However, a number of cellulosic fibers 232 are adjacent and partially wrapping around the synthetic polymer fibers 242, but there is not thermal bond between the cellulosic fibers 232 and the synthetic polymer fibers 242.

Because there is little or no bonding between the cellulosic fibers and the synthetic polymer fibers in this product, it can be expected that desirable characteristics and capabilities that would result from such bonding would be present only to a lesser extent, if at all, in such a product. However, because the synthetic polymer fibers are generally bonded together at locations throughout the material, and because there is not a pattern of unbonded regions, the inventors of the present invention theorize (without intending to be bound by any theory) that this contributes to making the product stiffer and less soft.

Further, it appears from FIG. 17 that essentially the entire length of each synthetic polymer fiber has melted and resolidified so that there is a significantly increased possibility for creating thermal bonds everywhere along its length where it may contact another synthetic polymer fiber. However, notwithstanding such extensive melting, the creation of significant bonding between the synthetic polymer fibers and adjacent cellulosic fibers is minimal or substantially non-existent. Without intending to be bound by any particular theory, the inventors of the present invention theorize that a material which does not have significant bonding between synthetic polymer fibers and cellulosic fibers will have a greater tendency to exhibit lower integrity and have more pulp dust.

In contrast, the material of the present invention has good structural integrity and minimal dust release while still remaining relatively soft and supple, and these desirable characteristics are exhibited by the material of the present invention without the use of chemical binders.

It will be readily apparent from the foregoing detailed description of the invention and from the illustrations thereof that numerous variations and modifications may be effected without departing from the true spirit and scope of the novel concepts or principles of the invention.

What is claimed is:

1. An absorbent material comprising a compacted web of cellulosic fibers and synthetic polymer fibers, wherein
at least the major portion of the total surface area defined on the exteriors of said synthetic polymer fibers has not been melted and resolidified; and
said web has a Gurley Stiffness of less than about 1500 milligrams.

2. The absorbent material in accordance with claim 1 in which
said web is substantially free of added chemical binders;
said web has a density between about 0.25 grams per cubic centimeter and about 0.50 grams per cubic centimeter; and
said web has a basis weight between about 100 grams per square meter and about 650 grams per square meter.

3. The absorbent material in accordance with claim 1 in which said web has
(A) a first, second, and third fluid acquisition rate of at least 1 milliliter per second, 1.25 milliliter per second, and 1.07 milliliter per second, respectively;
(B) a first, second, and third rewet of less than 0.03 grams, 0.1 grams, and 9 grams, respectively; and
(C) a normalized wicking energy of at least 1,800 ergs per gram.

4. The absorbent material in accordance with claim 1 in which said web
(A) includes a carrier layer exposed on one side of said web;
(B) includes an exposed cover layer on the side of the web opposite said carrier layer; and
(C) has been embossed with a pattern of surface indentations on at least one side between a pair of rolls each having a temperature of at least 120° C.

5. The absorbent material in accordance with claim 1 in which said synthetic polymer fibers comprise at least one of polypropylene fibers, polyethylene terephthalate fibers, and bicomponent fibers.

6. The absorbent material in accordance with claim 1 in which said web includes
(A) superabsorbent material in an amount which is between about 10% and 60% by weight of said web; and
(B) said synthetic polymer fibers in an amount which is at least about 5% by weight of said web.

7. The absorbent material in accordance with claim 1 in which liquid stable bonds are formed between said cellulosic fibers and said synthetic polymer fibers.

8. An absorbent material comprising a compacted web of cellulosic fibers and synthetic polymer fibers, wherein
at least some of said synthetic polymer fibers and cellulosic fibers are joined by liquid stable bonds; and
said web has a Gurley Stiffness of less than about 1500 milligrams.

9. The absorbent material in accordance with claim 8 in which
said web is substantially free of added chemical binders;
said web has a density between about 0.25 grams per cubic centimeter and about 0.50 grams per cubic centimeter; and
said web has a basis weight between about 100 grams per square meter and about 650 grams per square meter.

10. The absorbent material in accordance with claim 8 in which said web has
(A) a first, second, and third fluid acquisition rate of at least 1 milliliter per second, 1.25 milliliter per second, and 1.07 milliliter per second, respectively;
(B) a first, second, and third rewet of less than 0.03 grams, 0.1 grams, and 9 grams, respectively; and
(C) a normalized wicking energy of at least 1,800 ergs per gram.

11. The absorbent material in accordance with claim 8 in which said web
(A) includes a carrier layer exposed on one side of said web;
(B) includes an exposed cover layer on the side of the web opposite said carrier layer; and
(C) has been embossed with a pattern of surface indentations on at least one side between a pair of rolls each having a temperature of at least 120° C.

12. The absorbent material in accordance with claim 8 in which
said synthetic polymer fibers comprise at least one of polypropylene fibers, polyethylene terephthalate fibers, and bicomponent fibers; and
said liquid stable bonds are formed at melted and resolidified portions of said synthetic polymer fibers in contact with said cellulosic fibers.

13. The absorbent material in accordance with claim 8 in which said web includes
(A) superabsorbent material in an amount which is between about 10% and 60% by weight of said web; and
(B) said synthetic polymer fibers in an amount which is at least about 5% by weight of said web.

14. The absorbent material in accordance with claim 8 in which at least the major portion of the surface area defined on the exteriors of said synthetic polymer fibers has not been melted and resolidified.

15. A process for making an absorbent material comprising the steps of:
(A) forming a web of cellulosic fibers and synthetic polymer fibers; and
(B) moving said web between a pair of heated rolls to compact said web while maintaining each of said rolls at temperatures to form liquid stable bonds that (1) are located at least between said cellulosic fibers and said synthetic polymer fibers, and (2) are insufficient to create a web Gurley Stiffness of more than about 1500 milligrams.

16. The process in accordance with claim 15 in which step (B) includes (1) moving said web at a selected speed, (2) compacting said web under a selected compaction load to a density of between about 0.25 grams per cubic centimeter and 0.50 grams per cubic centimeter, and (3) maintaining each of said rolls at temperatures which are insufficient at said selected speed of web movement and said selected compaction load to melt a major portion of the total surface area defined by the exteriors of said synthetic polymer fibers.

17. The process in accordance with claim 15 in which step (B) includes maintaining each of said rolls at temperatures such that only a minor portion of the total surface area defined by the exteriors of said synthetic polymer fibers is melted.

18. The process in accordance with claim 15 in which step (A) includes
providing superabsorbent material as part of said web in an amount which is between about 10% and 60% by weight of said web;
providing said synthetic polymer fibers in an amount which is at least about 5% by weight of said web; and
forming said web substantially free of added chemical binders.

19. The process in accordance with claim 15 in which step (B) includes
embossing a pattern of surface indentations into one side of said web with one of said rolls while maintaining each of said rolls at a temperature of at least 120°;
maintaining said selected speed of web movement at between about 30 meters per minute and about 300 meters per minute; and
maintaining said selected compaction load between about 28 newtons per millimeter of transverse web width to about 400 newtons per millimeter of transverse web width.

20. The process in accordance with claim 15 in which step (A) includes forming said web with a basis weight between about 100 grams per square meter and about 650 grams per square meter.
